(19) United States
(12) Patent Application Publication
(10) Pub. No.: US 2020/0281976 A1
(43) Pub. Date: Sep. 10, 2020

(54) PREVENTION AND TREATMENT OF GVHD AND AUTOIMMUNE DISEASES

(71) Applicant: CITY OF HOPE, Duarte, CA (US)

(72) Inventors: Defu ZENG, Arcadia, CA (US); Arthur RIGGS, Duarte, CA (US)

(73) Assignee: CITY OF HOPE, Duarte, CA (US)

(21) Appl. No.: 16/753,510

(22) PCT Filed: Oct. 4, 2018
(86) PCT No.: PCT/US2018/054446

§ 371 Date: Apr. 3, 2020

Related U.S. Application Data
(60) Provisional application No. 62/568,204, filed on Oct. 4, 2017.

(51) Int. Cl. A61K 35/17; C07K 16/28; A61P 37/06

(52) U.S. Cl. CPC A61K 35/17; C07K 16/2815; A61K 2039/505; C07K 16/2812; A61P 37/06; C07K 16/2809

(57) ABSTRACT

Disclosed herein are methods of preventing and treating GVHD and autoimmune diseases. The methods entail administering one or more doses of an effective amount of a therapeutic agent to a subject to in vivo knock down Stat3 in the T cells and/or B cells of the subject. Alternatively, the methods entail contacting donor T cells and/or B cells with an effective amount of a therapeutic agent to in vitro knock down Stat3 and administering the Stat3-deficient T cells and/or B cells to the subject. Some examples of the therapeutic agent include small molecule Stat3 inhibitors such as Stat3 siRNAs delivered by an antibody to specifically knock down Stat3 in the lymphocytes of the target tissue.

### PRIORITY CLAIM

This application claims priority to U.S. Provisional Application No. 62/568,204, filed Oct. 4, 2017, which is incorporated by reference herein in its entirety, including drawings.

### STATEMENT OF GOVERNMENT INTEREST

The present invention was made with government support under Grant No. RO1 AI066008, R56-AI066008, and P30CA033572, awarded by the National Institutes of Health (NIH). The Government has certain rights in the invention.

### BACKGROUND

Allogeneic hematopoietic cell transplantation (HCT) is a curative therapy for hematological malignancies certain hereditary disorders, and refractory autoimmune diseases¹. Chronic graft-versus-host disease (cGVHD) remains a major obstacle to the success of this treatment^{2,3}. Chronic GVHD presents with multi-organ pathology and common diagnostic features, as outlined by the NIH consensus criteria. Manifestations include skin pathology varying from lichen planus-like lesions to extensive cutaneous sclerosis, bronchiolitis obliterans as well as salivary and lacrimal gland pathology⁴. Chronic GVHD is an autoimmune-like syndrome caused by the interactions of donor CD4⁺ T and B cells and production of IgG^{2,5-9}. Chronic GVHD often follows acute GVHD. The pathogenic autoreactive CD4⁺ T cells in cGVHD can derive from CD4⁺ T cells in the graft or from T cells generated de novo in a thymic environment damaged by acute GVHD⁷. Due to the destructive effect of alloreactive and autoreactive T cells and IgG antibodies, cGVHD recipients often have lymphopenia at the disease onset⁹⁻¹¹. This feature differs from other autoimmune diseases (for example, systemic lupus, multiple sclerosis, and type 1 diabetes) that usually have increased numbers of lymphocytes in lymphoid tissues at disease onset¹².

Accordingly, there remain needs to develop an effective way to prevent and treat GVHD as well as other autoimmune diseases. This invention satisfies the needs in the art.

### SUMMARY

In one aspect, the disclosure provided herein relates to a method of preventing a subject from suffering from GVHD or treating a subject suffering from GVHD after hematopoietic cell transplantation (HCT) while preserving GVL. In some embodiments, the GVHD is chronic GVHD. In some embodiments, the method entails administering one or more doses of an effective amount of a therapeutic agent to the subject simultaneously, immediately before, or immediately after HCT to in vivo knock down Stat3 in recipient T cells and/or B cells. Alternatively, the method entails contacting donor T cells and/or B cells with an effective amount of a therapeutic agent to knock down Stat3 in vitro, and administering the Stat3-deficient T cells and/or B cells to the subject. In some embodiments, the therapeutic agents include, but are not limited to, Stat3 siRNAs or other small molecule Stat3 inhibitors delivered by antibodies such as an anti-CD4 antibody, an anti-CD8 antibody, an anti-CD3 antibody, and an anti-CD19 antibody to specifically knock down Stat3 in T cells and/or B cells in lymphoid tissue and/or target tissue. In some embodiments, the antibody is a monoclonal antibody or a humanized antibody. In some embodiments, Stat3 siRNAs are delivered in vitro by lentivirus, adenovirus, electroporation, or zinc finger nucleases. In some embodiments, the therapeutic agent specifically targets Stat3 in CD4⁺ T cells and knocks down Stat3 in CD4⁺ donor or recipient T cells. In some embodiments, the method further entails a step of administering thymic progenitors including embryonic stem (ES) cells and induced pluripotent stem (iPS) cells to the subject to reestablish thymus activity in the subject.

In one aspect, the disclosure provided herein relates to a method of knocking down Stat3 in T cells and/or B cells in vivo by administering one or more doses of an effective amount of a therapeutic agent to the subject to knock down Stat3 in the T cells and/or B cells. Also disclosed is a method of knocking down Stat3 in T cells and/or B cells in vitro by contacting donor T cells and/or B cells with an effective amount of a therapeutic agent to knock down Stat3 in the donor T cells and/or B cells. In some embodiments, the therapeutic agents include, but are not limited to, Stat3 siRNAs or other small molecule Stat3 inhibitors delivered by antibodies, such as an anti-CD4 antibody, an anti-CD8 antibody, an anti-CD3 antibody, and an anti-CD19 antibody to specifically knock down Stat3 in T cells and/or B cells in lymphoid tissue and/or target tissue. In some embodiments, the antibody is a monoclonal antibody or a humanized antibody. In some embodiments, Stat3 siRNAs are delivered in vitro by lentivirus, adenovirus, electroporation, or zinc finger nucleases. In some embodiments, the therapeutic agent specifically targets Stat3 in CD4⁺ T cells and knocks down Stat3 in CD4⁺ donor or recipient T cells.

In another aspect, the disclosure provided herein relates to a method of preventing or treating a subject who is at a risk of or who is suffering from an autoimmune disease. In some embodiments, the autoimmune disease includes Sjogren's syndrome, systemic lupus erythematosus, rheumatoid arthritis, type 1 diabetes, multiple sclerosis, and aplastic anemia. In some embodiments, the method entails administering one or more doses of an effective amount of a therapeutic agent to a subject to in vivo knock down Stat3 in the T cells and/or B cells of the subject. Alternatively, the method entails contacting donor T cells and/or B cells with an effective amount of a therapeutic agent to knock down Stat3 in vitro, and administering the Stat3-deficient T cells and/or B cells to the subject. In some embodiments, the therapeutic agents include, but are not limited to, Stat3 siRNAs or other small molecule Stat3 inhibitors delivered by antibodies such as an anti-CD4 antibody, an anti-CD8 antibody, an anti-CD3 antibody, and an anti-CD19 antibody to specifically knock down Stat3 in T cells and/or B cells in lymphoid tissue and/or target tissue. In some embodiments, Stat3 siRNAs are delivered in vitro by lentivirus, adenovirus, electroporation, or zinc finger nucleases. In some embodiments, the antibody is a monoclonal antibody or a humanized antibody. In some embodiments, the therapeutic agent specifically targets Stat3 in CD4⁺ T cells and knocks down Stat3 in CD4⁺ donor or recipient T cells.

### BRIEF DESCRIPTION OF THE DRAWINGS

This application contains at least one drawing executed in color. Copies of this application with color drawing(s) will be provided by the Office upon request and payment of the necessary fees.

FIG. 1 illustrates the potential function of PSGL-1^{lo}CD4⁺ T and B interactions in the pathogenesis of chronic GVHD.

FIGS. 2A-2I show the gating strategies for flow cytometry analysis. Representative gating strategies to analyzes: FIG. 2A: percentage of Tfh shown in FIGS. 11F, 7F and 18. FIG. 2B: percentage of germinal centre B shown in FIGS. 11G, 7G. FIG. 2C: percentage of PSGL-1^{lo}CD4⁺ T shown in FIGS. 15A, 15B, 22A, 23F, 34C, 16, 19, and 30, and sorting. FIG. 2D: binding of Anti-Rat IgG2b shown in FIG. 22C. FIG. 2E: binding of Anti-ICOS shown in FIG. 22D. FIG. 2F: ICOSL expression level on B cells shown in FIG. 22E. FIG. 2G: CD4⁺CD8⁺ thymocyte shown in FIGS. 34A, 34B, 4, 24, 25, and 28. FIG. 2H: CCR9 expression level shown in FIG. 34D. Similar gating strategy to FasL expression level shown in FIG. 34D. FIG. 2I: percentage of Treg shown in FIG. 33.

FIGS. 3A-3E show that no germinal center formation is observed in cGVHD recipients. BALB/c mice were irradiated (850 cGy) and given 2.5×10⁶ TCD-BM alone (n=6) or 2.5×10⁶ TCD-BM plus 1×10⁶ (n=8) or 0.01×10⁶ (n=8) splenocytes from C57BL/6 donors. Mice were monitored for cGVHD. FIG. 3A shows cutaneous cGVHD score (severe cGVHD group versus no-GVHD group: P<0.001, severe cGVHD group versus mild cGVHD group: P<0.001, two-way ANOVA). FIG. 3B shows picture taken on day 60 after transplantation (1—no GVHD, 2—mild cGVHD, 3—severe cGVHD). FIG. 3C shows survival curve (severe cGVHD group versus no GVHD group: P<0.001, severe cGVHD group versus mild cGVHD group: P<0.001, log-rank test). FIG. 3D shows immunofluorescent staining of B220, CD3, and PNA on cryosections of spleen harvested on day 60 after HCT. FIG. 3E shows that germinal center number and area were measured and are shown as mean±SE (n=6). Scale bar, 50 μm.

FIG. 4 shows representative flow patterns of thymocytes in recipients with different severity of cGVHD. BALB/c mice were irradiated (850 cGy) and given 2.5×10⁶ TCD-BM (T cell depleted bone marrow) alone (n=4) or 2.5×10⁶ TCD-BM plus 1×10⁶ (n=4) or 0.01×10⁶ (n=4) splenocytes from C57BL/6 donors. 60 days after transplantation thymus specimens were harvested and stained with CD4 and CD8. Gated mononuclear cells are shown as CD4 versus CD8. One representative of 4 recipients in each group is shown.

FIG. 5 shows that no germinal centers were observed in BALB/c recipients on day 15 after HCT. BALB/c mice were irradiated (850 cGy) and given 2.5×10⁶ TCD-BM alone or 2.5×10⁶ TCD-BM plus 1×10⁶ or 0.01×10⁶ (n=6) splenocytes from C57BL/6 donors. Immunofluorescent staining pattern of B220, CD3 and PNA on cryosections of spleen harvested at 15 days after transplantation. One representative of 6 recipients in each group is shown. Scale bar, 50 μm.

FIG. 6 shows that germinal center formation was not observed in BALB/c recipients with severe GVHD. BALB/c mice were irradiated (850 cGy) and given 2.5×10⁶ TCD-BM alone or 2.5×10⁶ TCD-BM plus 1×10⁶ or 0.01×10⁶ (n=6) splenocytes from C57BL/6 donors. Immunofluorescent staining of B220, CD3 and PNA on cryosections of spleen harvested at 30 days after transplantation. One representative of 6 recipients in each group is shown. Scale bar, 50 μm.

FIGS. 7A-7G show no germinal center formation in chronic GVHD C57BL/6 recipients given LP/J grafts. Lethally irradiated C57BL/6 recipients were given whole spleen (10×10⁶) and BM (2.5×10⁶) from LP/J donors. Recipients were monitored for cGVHD for up to 60 days (N=12). FIG. 7A shows cutaneous GVHD scores (Group 3 vs Group 1: P<0.001, Group 3 vs Group 2: P<0.001, two-way ANOVA test). FIG. 7B shows survival curve. FIG. 7C shows a representative photograph of a GVHD-free recipient, 3 recipients with mild GVHD, and 3 recipients with facial hair-loss at 60 days after HCT. FIG. 7D shows one representative of 8 skin and salivary gland histopathology evaluation is shown. Scale bar, 50 μm. FIG. 7E shows that 60 days after HCT, splenic cryosections were analyzed by immunofluorescent staining for lymphoid follicle structure and GC formation. B220 (Green) and CD3 (red) were used to show the B and T cell zone of lymphoid follicle, respectively. Peanut Agglutinin (PNA, blue) was used to detect GC B cells. One representative of 4 recipients in each group is shown. Scale bar, 50 μm. FIG. 7F shows that splenocytes were stained for CD4, CD19, CXCR5, and PD-1. Gated donor CD4⁺CD19⁻ cells are shown as CXCR5 versus PD-1. CXCR5^{hi}PD-1^{hi} cells were gated as TFH. Percentages of CXCR5^{hi}PD-1^{hi} cells among CD4⁺CD19⁻ cells are shown as mean±SE (N=4). FIG. 7G shows that splenocytes were stained with CD19, Fas and GL7. Gated CD19⁺ cells are shown as GL7 versus Fas. GL7⁺Fas⁺ cells were gated as germinal center B cells. Percentages of GL7⁺Fas⁺ among CD19⁺ cells are shown as mean±SE (N=4). **P<0.01, ***P<0.001, unpaired 2-tailed student t test.

FIGS. 8A-8B show that different combination of staining reagents fail to detect GC formation in B10.BR recipients with severe chronic GVHD. Lethally irradiated B10.BR recipients were given 2.5×10⁶ TCDBM alone or 2.5×10⁶ TCDBM plus 1×10⁶ or 0.1×10⁶ splenocytes. 28 days after HCT, spleens were harvested. FIG. 8A shows that splenic cryosections were analyzed by immunofluorescent staining of lymphoid follicle structure and GC formation. B220 (Green) and CD3 (red) were used to show the B and T cell zones of lymphoid follicles, respectively. Peanut Agglutinin (PNA, blue) was used to detect GC B cells. FIG. 8B shows that IgM (Green) was used to show the B cell zone of lymphoid follicles. Peanut Agglutinin (PNA, blue) was used to detect GC B cells. One representative of 4 recipients in each group is shown. Scale bar, 50 μm.

FIGS. 9A-9B show that different combination of staining reagents fail to detect GC formation in C57BL/6 recipients with severe chronic GVHD. Lethally TBI-conditioned C57BL/6 mice were given with whole spleen (10×10⁶) and TCDBM (2.5×10⁶) from LP/J donors. 60 days after HCT, splenic sections of recipients were analyzed by immunofluorescent staining for lymphoid follicle structure and GC formation. FIG. 9A that IgM (Green) was used to show the B cell zone of lymphoid follicle. Peanut Agglutinin (PNA, blue) was used to detect GC B cells. FIG. 9B shows that IgD (Green) was used to show the B cell zone of lymphoid follicles. Gly7 (blue) was used to detect GC B cells. One representative of 4 recipients in each group is shown. Scale bar, 50 μm.

FIGS. 10A-10E shows that no germinal center formation in BALB/c recipients given MHC-matched B10.D2 grafts. Lethally irradiated BALB/c mice were transplanted with whole spleen (10×10⁶) and TCDBM (2.5×10⁶) from B10.D2 donors. The recipients were monitored for cGVHD development for up to 60 days (N=12). FIG. 10A shows cutaneous GVHD score (Group 2 vs Group 1: P<0.001 two-way ANOVA). FIG. 10B shows a representative photograph of 1 GVHD-free and 3 cGVHD recipients at day 60 after HCT. FIG. 10C shows the percentage of survival of recipients. FIG. 10D shows one representative skin and salivary gland histopathology. FIG. 10E shows that B220 (green) and CD3 (red) were used to show the B and T cell zones of lymphoid follicles, respectively. Peanut Agglutinin (PNA, blue) was used to detect GC B cells. One representative of 4 recipients in each group is shown. Scale bars, 50 μm.

FIGS. 11A-11G show that chronic GVHD was induced in recipients without germinal center formation. BALB/c recipients were irradiated (850 cGy) and given 2.5×10⁶ TCD-BM alone (n=12) or 2.5×10⁶ TCD-BM plus 1×10⁶ (n=12) splenocytes from either WT C57BL/6 or BCL6^{fl/fl} Mb1-Cre C57BL/6 donors. cGVHD development was monitored. FIG. 11A shows cutaneous cGVHD score. FIG. 11B shows picture taken at day 60 after HCT (1 and 3B-BCL6+/+no GVHD and cGVHD, 2 and 4B-BCL6^{-/-} no GVHD and cGVHD). FIG. 11C shows percentage of body weight changes. FIG. 11D shows survival curve. FIG. 11E shows that sixty days after transplantation, spleens were harvested and germinal centers were identified by immunofluorescent staining of B220, CD3, and PNA, and GC area and numbers were measured and are shown as mean±SE (n=6). FIG. 11F shows that donor splenocytes were stained for CD4, CD19, CXCR5, and PD-1. Tfh were gated as CD4⁺CD19⁻ and are shown as CXCR5^{hi}PD-1 hi. Percentages of CXCR5^{hi}PD-1 hi cells among CD4⁺CD19⁻ cells were shown as mean±SE (n=6). FIG. 11G shows that donor splenocytes were stained for CD19, GL7, and Fas. Germinal center B cells were gated on CD19⁺ and are shown as GL7⁺Fas⁺. Percentages of GL7⁺Fas⁺ among CD19⁺ cells are shown as mean±SE (n=6). **P<0.01, ***P<0.001, unpaired two-tailed Student's t test. B-BCL6^{+/+} no GVHD=B-BCL6^{+/+} TCD-BM; B-BCL6^{-/-} no GVHD=B-BCL6^{-/-} TCD-BM; B-BCL6^{+/+} cGVHD=B-BCL6^{+/+} TCD-BM+B-BCL6^{+/+} splenocytes; B-BCL6^{-/-} cGVHD=B-BCL6^{-/-} TCD-BM+B-BCL6^{-/-} splenocytes. Scale bar, 50 μm.

FIG. 12 shows histopathology of salivary gland, skin, lung and liver of BALB/c recipients given grafts with BCL6^{-/-} B cells. BALB/c recipients were irradiated with 850 cGy and given 2.5×10⁶ TCDBM alone or 2.5×10⁶ TCDBM plus 1×10⁶ splenocytes from either WT or B-BCL6^{-/-} C57BL/6 donors. cGVHD was monitored. 60 days after HCT, salivary gland, skin lung and liver specimens were harvested and used for H&E staining. One representative of 6 recipients in each group is shown. Scale bar, 50 μm.

FIG. 13 shows severe thymus damage in cGVHD recipients given B-BCL6^{-/-} grafts. 60 days after HCT, thymus specimens were harvested and stained with CK8 for cortex epithelial cells and UEA-1 for medulla epithelial cells. One representative of 6 recipients in each group is shown. Scale bar, 50 μm.

FIGS. 14A-14B show flow cytometry analysis of follicular B, T2 B, and T1 plus marginal zone B cells in recipients given B-BCL6^{-/-} grafts. FIG. 14A shows that 60 days after HCT, splenocytes were stained for CD23, IgM, IgD and CD21. Gated CD23⁺ cell is shown as IgM versus IgD. Gated IgM^{hi}IgD^{hi} cells are shown as IgM versus CD21. Percentages of CD23⁺, T2B (CD23⁺IgD^{hi}IgM^{hi}CD21⁺) cell and follicular B cells (CD23⁺IgD^{hi}IgM^{lo}CD21⁺) cells are shown as mean±SE (n=6) FIG. 14B shows gated CD23⁻ cells as IgM versus CD21. Percentages of T1+M2 cells (CD23-IgD^{lo}IgM^{hi}) are shown as mean±SE (n=6).

FIGS. 15A-15D show that cGVHD is associated with expansion of PSGL-1^{lo}CD4⁺ T cells. BALB/c recipients were irradiated (850 cGy) and given 2.5×10⁶ TCD-BM or 2.5×10⁶ TCD-BM plus 1×10⁶ splenocytes. FIGS. 15A and 15B show that 21, 30, and 45 days after HCT, spleen and lung were harvested. Splenocytes and mononuclear cells isolated from lung were stained for CD4, CD44, PSGL-1, and CD62L. Gated CD4⁺CD44^{hi} are shown as PSGL-1 versus CD62L. PSGL-1 low and CD62L low cells were gated as extrafollicular CD4⁺ T cells. Percentages of PSGL-1^{lo}CD62L^{lo} cells among CD4⁺CD44^{hi} cells are shown as mean±SE (n=8). FIG. 15C shows that 21 days after HCT, splenocytes from no-GVHD or cGVHD recipients given wild-type C57BL/6 transplants were harvested and stained for CD4, CD44, PSGL-1, and CD62L. CD44^{hi}CD62L^{lo}PSGL-1^{lo}CD4⁺ T cells were sorted and used for RNA isolation and RNA-Seq microarray analysis. Heat maps of RNA expression of CXCR4, CXCR5, and CCR7 are shown as mean centered log₂ expression. RNA-Seq microarray measurements were performed on duplicate samples from no-GVHD group and cGVHD group. Each sample represents splenocytes from eight recipients. FIG. 15D shows that 21 days after HCT, sorted CD4⁺CD44^{hi}PSGL-1^{lo}CD62L^{lo} cells were stimulated with PMA and ionomycin for 24 h. Stimulated cells were stained and are shown as CD4 versus IFN-γ, IL-13, IL-17, or IL-21. Percentages of CD4⁺IFN-γ⁺, CD4⁺IL-13+, CD4⁺IL-17+, or CD4⁺IL-21⁺ cells among CD4⁺ T cells are shown as mean±SE (n=9). *P<0.05, ***P<0.001, unpaired two-tailed Student's t test.

FIG. 16 shows CD4⁺CD44^{hi}CD62L^{lo}PSGL-1^{lo} T cell population in the liver of cGVHD BALB/c recipients. BALB/c recipients were irradiated (850 cGy) and given 2.5×10⁶ TCDBM or 2.5×10⁶ TCDBM plus 1×10⁶ splenocytes. 21, 30, 45 days after HCT, mononuclear cells isolated from liver were stained for CD4, CD44, PSGL- and CD62L. Gated CD4⁺CD44^{hi} are shown as PSGL-1 versus CD62L. Percentage of PSGL-1^{lo}CD62L^{lo} cells among CD4⁺CD44^{hi} cells are shown as mean±SE (n=8). **p<0.01, *** p<0.001 unpaired 2-tailed student t test.

FIG. 17 shows CXCR4, CXCR5 and CCR7 mRNA levels tested by real time PCR. 21 days after HCT, splenocytes from no GVHD or chronic GVHD recipients given wild-type C57BL/6 donors were harvested and stained for CD4, CD44, PSGL-1 and CD62L. CD44^{hi}CD62L^{lo}PSGL-1^{lo}CD4⁺ T cells were sorted and used for RNA isolation. mRNA level of CXCR4, CXCR5 and CCR7 was measured by real time PCR. Mean±SE is shown of 4 replicate experiments. Each experiment is combined from 8 mice, ***p<0.001 unpaired 2-tailed student t test.

FIG. 18 shows that no expansion of Tfh was observed in BALB/c recipients given MHC mismatched C57BL/6 grafts 21 days after HCT. Lethally irradiated BALB/c recipients were given whole spleen (1×10⁶) and TCD-BM (2.5×10⁶) from C57BL/6 donors. 21 days after HCT splenocytes were stained for CD4, CD19, CXCR5 and PD-1. Tfh were gated as CD4⁺CD19⁻ and are shown as CXCR5^{hi}PD-1^{hi}. Percentages of CXCR5^{hi}PD-1^{hi} cells among CD4⁺CD19⁻ cells were shown as mean±SE (n=6). NS, unpaired 2-tailed student t test.

FIGS. 19A-19B show that expansion of PSGL-1^{lo}CD4⁺ T cells in cGVHD recipients is driven alloimmune responses. Lethally irradiated C57BL/6 recipients were given whole spleen (10×10⁶) plus TCD-BM (2.5×10⁶) from MHC-matched but minor mismatched LP/J donors (FIG. 19A) or from syngeneic C57BL/6 donors (FIG. 19B). 21 days after HCT, splenocytes were stained for CD4, CD44, PSGL-1, and CD62L. Gated CD4⁺CD44^{hi} are shown as PSGL-1 versus CD62L. PSGL-1 low and CD62L low cells were gated as extrafollicular CD4⁺ T cells. Percentages of PSGL-1^{lo}CD62L^{lo} cells among CD4⁺CD44^{hi} cells are shown as mean±SE (n=6). ***p<0.001, NS unpaired 2-tailed student t test.

FIGS. 20A-20F show that anti-ICOS treatment ameliorates cGVHD in recipients without GC formation. FIG. 20A shows that as illustrated in FIG. 15, 21 days after HCT, plenocytes from no-GVHD or cGVHD recipients given wild-type C57BL/6 donors were harvested and stained for CD4, CD44, PSGL-1, and CD62L. CD44^{hi}CD62L^{lo}PSGL-1^{lo}CD4⁺ T cells were sorted and used for RNA isolation and RNA-Seq microarray analysis. Heat maps of RNA expression levels of costimulatory and coinhibitory markers are shown as mean centered log₂ expression. RNA-Seq microarray measurements were performed on duplicate samples from no-GVHD group and cGVHD group. Each sample represents splenocytes from eight recipients. FIGS. 21B-21F show that BALB/c recipients were irradiated (850 cGy) and given either 2.5×10⁶ TCD-BM (n=8) or 2.5×10⁶ TCD-BM plus 1×10⁶ splenocytes (n=12) from B-BCL6^{-/-} C57BL/6 donors. Recipients given 2.5×10⁶ TCD-BM plus 1×10⁶ splenocytes were treated with anti-ICOS or isotype control of rat IgG2b, 200 μg/mouse i.p., starting on day 0 and repeated every other day until day 45 after HCT. Chronic GVHD was monitored. FIG. 21B shows cutaneous cGVHD score (Group 3 versus Group 2: P<0.001 two-way ANOVA). FIG. 21C shows picture taken on day 60 after HCT (1—no-GVHD, 2—isotype, 3—anti-ICOS). FIG. 21D shows survival curve (Group 3 versus Group 2: P<0.05, log-rank test). FIG. 21E shows H&E staining of salivary gland, skin, lung, and liver. FIG. 21F shows pathology scores of cGVHD for salivary gland, skin, lung, and liver as mean±SE (n=6). **P<0.01, unpaired two-tailed Student's t test. Scale bar, 50 μm.

FIG. 21 shows mRNA levels of ICOS, PD1, PD-L1 and CD80 tested by real time PCR. 21 days after HCT, splenocytes from no GVHD or chronic GVHD recipients given wild-type C57BL/6 donors were harvested and stained for CD4, CD44, PSGL-1 and CD62L. CD44^{hi}CD62L^{lo}PSGL-1^{lo}CD4⁺ T cells were sorted and used for RNA isolation. mRNA level of was measured by real time PCR. Mean±SE is shown of 4 replicate experiments. Each experiment is combined from 8 mice. *p<0.05, **p<0.01, *** p<0.001 unpaired 2-tailed student t test.

FIGS. 22A-22E show that anti-ICOS treatment reduces PSGL-1^{lo}CD4⁺ T-cell expansion. As illustrated in FIG. 20, BALB/c recipients were irradiated (850 cGy) and given 2.5×10⁶ TCD-BM plus 1×10⁶ splenocytes. Recipients were treated with anti-ICOS or control rat IgG2b (200 μg/mouse i.p.) every other day from day 0 to day 45 after HCT. FIG. 22A shows that 21 days after HCT, mononuclear cells from spleen, lung, and liver were stained for CD4, CD44, PSGL-1, and CD62L. Gated CD4⁺CD44^{hi} cells are shown as PSGL-1 versus CD62L (n=8). FIG. 22B shows that serum anti-dsDNA was measured at 45 days after HCT. FIGS. 22C-22E show that 21 days after HCT, mononuclear cells from spleen, lung, and liver were stained with CD4, anti-rat IgG2b, or anti-ICOS, or stained with anti-CD19 and anti-ICOSL. Gated CD4⁺ T cells are shown as anti-rat IgG2b (FIG. 22C) or anti-ICOS (FIG. 22D) staining. FIG. 22E shows gated CD19⁺ B cells as ICOSL staining. One representative of four experiments is shown. **P<0.01, ***P<0.001, unpaired two-tailed Student's t test.

FIGS. 23A-23F show that BCL6 deficiency in donor CD4⁺ T cells prevents expansion of PSGL-1^{lo}CD4⁺ T cells and cutaneous cGVHD. BALB/c recipients were irradiated (850 cGy) and given 2.5×10⁶ TCD-BM alone or 2.5×10⁶ TCD-BM plus 1×10⁶ splenocytes from either WT or B-BCL6^{-/-} C57BL/6 donors (n=12). cGVHD was monitored. FIG. 23A shows percentage of body weight changes. FIG. 23B shows cutaneous cGVHD scores (Group 3 versus Group 2: P<0.001 two-way ANOVA). FIG. 23C shows picture taken at day 60 after HCT (1 and 2-CD4-BCL6^{+/+} no GVHD and GVHD, 3CD4-BCL6^{-/-} GVHD). FIG. 23D shows survival curve. FIG. 23E shows that 60 days after transplantation, spleens were harvested, and germinal centers were identified by immunofluorescent staining of B220, CD3, and PNA. GC area and numbers were measured and are shown as mean±SE (n=4). FIG. 23F shows that 21 days after HCT, mononuclear cells from spleen, lung, and liver were stained for CD4, CD44, PSGL-1, and CD62L. Gated CD4⁺CD44^{hi} cells are shown as PSGL-1 versus CD62L. Percentages of CD62L^{lo}PSGL-1^{lo} cells among CD4⁺CD44^{hi} are shown as mean±SE (n=8). ***P<0.001, unpaired two-tailed Student's t test. CD4-BCL6^{+/+} no-GVHD=CD4-BCL6^{+/+} TCD-BM; CD4-BCL6^{-/-} no-GVHD=CD4-BCL6^{-/-} TCD-BM; CD4-BCL6^{+/+} cGVHD=CD4-BCL6^{+/+} TCD-BM+CD4-BCL6^{+/+} splenocytes; CD4-BCL6^{-/-} cGVHD=CD4-BCL6^{-/-} TCD-BM+CD4-BCL6^{-/-} splenocytes. Scale bar, 50 μm.

FIGS. 24A-24B show little skin damage and reduced thymus damage in recipients given CD4-BCL6^{-/-} grafts. BALB/c recipients were irradiated (850 cGy) and given 2.5×10⁶ TCDBM alone or 2.5×10⁶ TCDBM plus 1×10⁶ splenocytes from either WT or CD4-BCL6^{-/-} C57BL/6 donors. FIG. 24A shows that 60 days after HCT, salivary gland, skin, lung and liver specimens were harvested and used for H&E staining. One representative of 6 recipients in each group is shown. FIG. 24B shows that 60 days after transplantation, thymus specimens were harvested and stained for CD4 and CD8. Gated mononuclear cells are shown as CD4 versus CD8. One representative of 6 recipients in each group is shown.

FIGS. 25A-25D show that adoptive transfer of extrafollicular PSGL-1^{lo}CD4⁺ T cells augments cutaneous chronic GVHD. BALB/c recipients were conditioned with 850 cGy TBI and given TCD-BM (2.5×10⁶) alone or TCDBM plus splenocytes (1×10⁶) from either B-BCL6^{-/-} or CD4-BCL-6^{-/-} C57BL/6 donors. 21 days after transplantation, sorted PSGL1^{lo}CD4⁺ T cells from GVHD recipient given B-BCL6^{-/-} transplants were injected into recipients given CD4-BCL6^{-/-} transplants. Recipients given PBS were used as control. Recipients were monitored for chronic GVHD development for up to 60 days. FIG. 25A shows cutaneous cGVHD scores (Group 3 vs Group 2: P<0.001 two-way ANOVA). There were 8 mice per group combined from two replicate experiments. FIG. 25B shows picture taken at D60 after HCT, one representative is shown of 8. 1—no GVHD, 2—HCT buffer, 3—cGVHD ExT. FIG. 25C shows H&E staining of skin, one representative is shown of 6. FIG. 25D shows that 60 days after first transplantation, thymus specimens were harvested and stained for CD4 and CD8. Gated mononuclear cells are shown as CD4 versus CD8 (n=6) ***p<0.001 unpaired 2-tailed student t test.

FIGS. 26A-26G show that Stat3 deficiency in donor CD4⁺ T cells prevents expansion of PSGL-1^{lo}CD4⁺ T cells and systemic cGVHD. FIG. 26A shows that 21 days after HCT, splenocytes from no-GVHD or cGVHD recipients given wild-type C57BL/6 donors were harvested and stained for CD4, CD44, PSGL-1, and CD62L. CD44^{hi}CD62L^{lo}PSGL-1^{lo}CD4⁺ T cells were sorted and used for RNA isolation and RNA-Seq microarray analysis. Heat maps of RNA expression of transcription factor in extrafollicular T cells are shown as mean centered log₂ expression. RNA-Seq microarray measurements were performed on duplicate samples from no-GVHD and cGVHD groups. Each sample represents splenocytes from eight recipients. FIGS. 26B-26G show that BALB/c recipients were irradiated (850 cGy) and given 2.5×10⁶ TCD-BM alone or 2.5×10⁶ TCD-BM plus 1×10⁶ splenocytes (n=12) from either WT or CD4-STAT3^{-/-} donors. FIG. 26B shows percentage of body weight changes (Group 3 versus Group 4: P<0.001, two-way ANOVA). FIG. 26C shows cutaneous cGVHD scores (Group 3 versus Group 4: P<0.001, two-way ANOVA). FIG. 26D shows picture taken on day 60 after HCT (1 and 3—Stat3^{+/+}-no GVHD and cGVHD, 2 and 4—Stat3^{-/-}-no GVHD and cGVHD). FIG. 26E shows survival curve (Group 3 versus Group 4: P<0.05, log-rank test). FIG. 26F shows that 21 days after HCT, mononuclear cells from spleen, lung, and liver were stained with CD4, CD44, PSGL-1, and CD62L. Percentages of CD62L^{lo}PSGL-1^{lo} cells among CD4⁺CD44^{hi} are shown as mean±SE (n=6). FIG. 26G shows that 60 days after transplantation, spleens were harvested and germinal centers were identified by immunofluorescent staining of B220, CD3, and PNA, and GC area and numbers were measured and are shown as mean±SE (n=4). **P<0.01, ***P<0.001, unpaired two-tailed Student's t test. CD4-Stat3^{+/+} no GVHD=CD4-Stat3^{+/+} TCD-BM; CD4-Stat3^{-/-} no GVHD=CD4-Stat3^{-/-}TCD-BM; CD4-Stat3^{+/+} cGVHD=CD4-Stat3^{+/+} TCD-BM+CD4-Stat3^{+/+} splenocytes; CD4-Stat3^{-/-} cGVHD=CD4-Stat3^{-/-} TCD-BM+CD4-Stat3^{-/-} splenocytes. Scale bar, 50 μm.

FIG. 27 shows mRNA levels of STAT3 and BCL6 tested by real time PCR. 21 days after HCT, splenocytes from no GVHD or chronic GVHD recipients given wild-type C57BL/6 donors were harvested and stained for CD4, CD44, PSGL-1 and CD62L. CD44^{hi}CD62L^{lo}PSGL-1^{lo}CD4⁺ T cells were sorted and used for RNA isolation. mRNA level of Stat3 and BCL6 was measured by real time PCR. Mean±SE is shown of 4 replicate experiments. Each experiment is combined from 8 mice. *p<0.05, unpaired 2-tailed student t test.

FIGS. 28A-28B show no damage in thymus, skin and salivary gland in recipients given CD4-STAT3^{-/-} grafts. BALB/c recipients were irradiated (850 cGy) and given 2.5×10⁶ TCDBM alone or 2.5×10⁶ TCDBM plus 1×10⁶ splenocytes from either WT or CD4-STAT3^{-/-} C57BL/6 donors. FIG. 28A shows that 60 days after HCT, salivary gland, skin, lung and liver specimens were harvested and stained with H&E. One representative of 6 recipients in each group is shown. Scale bar, 50 μm. FIG. 28B shows that 60 days after transplantation, thymus specimens were harvested and stained for CD4 and CD8. Gated mononuclear cells are shown as CD4 versus CD8. One representative of 6 recipients in each group is shown.

FIG. 29 shows that the STAT3 deficient PSGL-1^{lo}CD4⁺ T expressed similar levels of BCL6 to wild type control. 21 days after HCT, spleen, were harvested from recipients given 2.5×10⁶ TCDBM plus 1×10⁶ from WT or CD4-STAT3^{-/-} C57BL/6 donors. Splenocytes were stained with CD4, CD44, PSGL-1 and CD62L. BCL6 expression on PSGL-1^{lo}CD4⁺ were shown. One representative of 6 recipients is shown.

FIG. 30 shows reduced PSGL-1^{lo}CD4⁺ T cells percentage in the spleen, lung, and liver of recipients given CD4-STAT3^{-/-} grafts. 21 days after HCT, spleen, lung and liver specimens were harvested from recipients given 2.5×10⁶ TCDBM alone or plus 1×10⁶ from WT or CD4-STAT3^{-/-} C57BL/6 donors. Splenocytes and mononuclear cells isolated from lung and liver were stained with CD4, CD44, PSGL-1 and CD62L. Gated CD4⁺CD44^{hi} are shown as PSGL-1 versus CD62L.

FIG. 31 shows serum anti-dsDNA levels of BALB/c recipients given wild-type or CD4-STAT3^{-/-} grafts. BALB/c recipients were irradiated (850 cGy) and given 2.5×10⁶ TCDBM plus 1×10⁶ splenocytes from wild-type, CD4-STAT3^{-/-} C57BL/6 donors. Serum anti-ds DNA levels from recipients 45 days after transplantation are shown as mean±SE (n=8). *** p<0.001 unpaired 2-tailed student t test.

FIGS. 32A-32B show no obvious IgG deposition in the skin and thymus of recipients given CD4-STAT3^{-/-} graft. BALB/c recipients were conditioned with 850 cGy TBI and transplanted with TCD-BM (2.5×10⁶) and spleen cells (1×10⁶) from WT or CD4-Stat3^{-/-} C57BL/6 donors. 45 days after HCT, skin and thymus tissues were stained for tissue IgG deposition with rat-anti-mouse IgG-FITC. In FIGS. 32A and 32B, upper rows are WT- and lower rows are CD4-Stat3^{-/-} C57BL/6 donors. One representative is shown of 6 recipients in each group.

FIG. 33 shows Treg in the spleen of recipients given grafts with Stat3^{+/+}, Stat3^{-/-} or BCL6^{-/-} CD4⁺ T cells. BALB/c recipients were irradiated (850 cGy) and given 2.5×10⁶ TCDBM alone or 2.5×10⁶ TCDBM plus 1×10⁶ splenocytes from either WT, CD4-STAT3^{-/-} or CD4-BCL6^{-/-} C57BL/6 donors. 45 days after transplantation, mononuclear cells from spleen, lung and liver were stained for CD4 and Foxp3. The flow cytometry patterns are shown as CD4 versus Foxp3. Percentages of CD4⁺Foxp3⁺ cells among CD4⁺ are shown as mean±SE (n=6). ***p<0.001 unpaired 2-tailed student t test.

FIGS. 34A-34D show that thymus recovery in recipients given Stat3^{-/-} transplants is associated with reduced PSGL-1^{lo}CD4⁺ T-cell infiltration in the thymus. BALB/c recipients were conditioned with 850 cGy TBI and given 2.5×10⁶ TCD-BM plus 1×10⁶ splenocytes from either WT or CD4-Stat3^{-/-} donors. FIGS. 34A and 34B show that 10 days (FIG. 34A) and 30 days (FIG. 34B) after HCT, thymus specimens were harvested and stained with CK8 for the cortex and UEA-1 for the medulla epithelial cells. Percentage of CD4⁺CD8⁺ thymocytes was measured with flow cytometry. FIG. 34C shows that 10 days after HCT, spleen and thymus were harvested and stained for CD4, CD44, PSGL-1, and CD62L. Gated CD4⁺CD44^{hi} are shown as PSGL-1 versus CD62L. PSGL-1^{lo}CD62L^{lo}CD4⁺CD44^{hi} cells were identified as extrafollicular PSGL1^{lo}CD4⁺ T cells. Percentages of PSGL-1^{lo}CD4⁺ T cells among CD4⁺CD44^{hi} cells are shown as mean±SE (n=6). FIG. 34D shows that CCR9 and FasL expression on splenic PSGL-1^{lo}CD4⁺ T cells were measured by flow cytometry and one representative histogram is shown (n=6). Scale bar, 50 μm.

FIG. 35 shows Stat3 knockdown by shRNA. Lentiviral vectors were transfected into HEK293 cells to measure human STAT3 (hSTAT3) knockdown, and mouse embryonic fibroblast (MEF) cells to measure mouse STAT3 (mSTAT3) knockdown. Normalized STAT3 to human GAPDH or mouse GAPDH ratio are shown. Three replicates in each experiment. Error bars represent standard derivation.

FIG. 36 shows body weight change of Stat3 knockdown mice after HCT. Lethally irradiated BALB/c recipients transplanted with sorted EGFP⁺ cells (1-2×10⁶) and TCD-BM (5×10⁶) from C57BL/6 donor. n=5-6 per group. Data referent mean±SEM combined from 2 independent experiments. Unpaired t test for Stat3 knockdown group and GFP control group (** P=0.0017).

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Chronic graft-versus-host disease (cGVHD) is an autoimmune-like syndrome mediated by pathogenic CD4⁺ T and B cells, but the function of extrafollicular and germinal center CD4⁺ T and B interactions in cGVHD pathogenesis remains largely unknown. As demonstrated in the working examples, extrafollicular CD4⁺ T and B interactions are sufficient for inducing cGVHD, while germinal center formation is dispensable. The pathogenesis of cGVHD is associated with the expansion of extrafollicular CD44^{hi}CD62^{lo}PSGL-1^{lo}CD4⁺ (PSGL-1^{lo}CD4⁺) T cells. These cells express high levels of ICOS, and the blockade of ICOS/ICOSL interaction prevents their expansion and ameliorates cGVHD. Expansion of PSGL-1^{lo}CD4⁺ T cells is also prevented by BCL6 or Stat3 deficiency in donor CD4⁺ T cells, with the induction of cGVHD ameliorated by BCL6 deficiency and completely suppressed by Stat3 deficiency in donor CD4⁺ T cells. These results support that Stat3- and BCL6-dependent extrafollicular CD4⁺ T and B interactions play important functions in the pathogenesis of cGVHD.

This disclosure relates to a method of preventing a subject from suffering from GVHD or treating a subject suffering from GVHD after hematopoietic cell transplantation (HCT) while preserving GVL. In particular, this method can prevent or treat chronic GVHD, which is commonly associated with HCT. The method entails in vivo knocking down Stat3 in the T cells and/or B cells of the subject who receives HCT (the recipient), in vitro knocking down Stat3 in the donor T cells and/or B cells, or both.

For in vivo knocking down Stat3, the method entails administering one or more doses of an effective amount of a therapeutic agent to the recipient simultaneously, immediately before, or immediately after HCT to knock down Stat3 in recipient T cells and/or B cells. When the therapeutic agent is administered immediately before HCT, a first dose of the therapeutic agent can be administered any time up to about 10 days before HCT. When the therapeutic agent is administered immediately after HCT, a first dose of the therapeutic agent can be administered any time up to about 6 weeks after HCT. In some embodiments, a first dose of the therapeutic agent is administered about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, or about 10 days, before HCT. In some embodiments, a first dose of the therapeutic agent is administered simultaneously with HCT. In some embodiments, a first dose of the therapeutic agent is administered about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, or about 6 weeks, after HCT.

Autoimmune diseases such as type 1 diabetes and systemic lupus often have autoantibodies before clinical onset of the disease. In fact, at this moment, pathogenic T cells has already start to expand and infiltrate target organs. Antibodies, including anti-CD3, CD4 or CD8 as well as anti-CD19 can be used to deliver Stat3 siRNA or other small molecule Stat3 inhibitors to specifically knockout Stat3 in the T cells and/or B cells. Stat3 siRNA or other small molecule Stat3 inhibitors can be delivered before clinical onset as a prevention, or can be delivered to treat the patients with clinical symptoms.

When multiple doses of one or more therapeutic agents are administered, it is within the purview of one of ordinary skill in the art to adjust the administration schedule to optimize the therapeutic effect. For example, one dose can be administered immediately before HCT, followed by additional doses administered during and/or immediately after HCT. In some embodiments, one or more doses of the therapeutic agent can be administered subsequently after the administration of the first dose, e.g., within one month of administration of the first dose. For example, the subsequent doses of the therapeutic agent can be administered in one-week intervals or in two-week intervals. The intervals between administrations can be optimized based on the therapeutic effects.

By administering one or more doses of the therapeutic agent, at least 50%, at least 60%, at least 70%, at least 80%, at least 90%, at least 95%, at least 98%, or at least 99% of Stat3 is knocked down in the recipient T cells and/or B cells.

In the alternative or in addition to in vivo knocking down Stat3 in recipient T cells and/or B cells, Stat3 in donor T cells and/or B cells can be knocked down by contacting the donor T cells and/or B cells with an effective amount of a therapeutic agent to knock down donor T cell and/or B cell Stat3 in vitro, and then administering the Stat3-deficient donor T cells and/or B cells to the recipient simultaneously, immediately before, or immediately after HCT. The Stat3-deficient donor T cells and/or B cells can be administered any time up to about 10 days before HCT, or any time up to about 6 weeks after HCT. In some embodiments, the Stat3-deficient donor T cells and/or B cells are administered about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, or about 10 days, before HCT. In some embodiments, the Stat3-deficient donor T cells and/or B cells are administered simultaneously with HCT. In some embodiments, the Stat3-deficient donor T cells and/or B cells are administered about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, or about 6 weeks, after HCT. Also, multiple doses of the Stat3-deficient donor T cells and/or B cells can be administered to the recipient at various intervals for an extended period of time before or after HCT to optimize the therapeutic effects.

The method disclosed herein can further include a step of administering thymic progenitors including embryonic stem (ES) cells and induced pluripotent stem (iPS) cells to the recipient to reestablish thymus activity in the subject.

In another aspect, this disclosure also relates to a method of preventing a subject from suffering from an autoimmune disease or treating a subject suffering from an autoimmune disease. Various autoimmune diseases that can be prevented or treated by the method disclosed herein include Sjogren's syndrome, systemic lupus erythematosus, and rheumatoid arthritis. The method entails in vivo knocking down Stat3 in the T cells and/or B cells of the subject suffering from or at the risk of an autoimmune disease, in vitro knocking down Stat3 in donor T cells and/or B cells and administering the Stat3-deficient donor T cells and/or B cells to the subject suffering from or at the risk of an autoimmune disease, or both.

In some embodiments, the method entails administering one or more doses of an effective amount of a therapeutic agent to a subject to in vivo knock down Stat3 in the T cells and/or B cells of the subject. Alternatively or in addition, the method entails contacting donor T cells and/or B cells with an effective amount of a therapeutic agent to knock down Stat3 in vitro, and administering the Stat3-deficient T cells and/or B cells to the subject.

This disclosure also provides a method of knocking down Stat3 in T cells and/or B cells in vivo or in donor T cells and/or B cells in vitro. The in vivo method entails administering one or more doses of an effective amount of a therapeutic agent to a subject to knock down Stat3 in the subject. The in vitro method entails contacting donor T cells and/or B cells with an effective amount of a therapeutic agent to knock down Stat3 in the donor T cells and/or B cells.

For all the methods disclosed herein, any therapeutic agent that effectively knock down Stat3 in T cells and/or B cells in vivo or in vitro can be used. For example, antibodies such as an anti-CD4 antibody, an anti-CD8 antibody, an anti-CD3 antibody, and an anti-CD19 antibody can be used to deliver Stat3 siRNAs or other small molecule Stat3 inhibitors to specifically knock down Stat3 in lymphocytes of the target tissue. Functional fragments of these antibodies can be used as long as the antibody fragments can effectively deliver Stat3 siRNAs or other small molecule Stat3 inhibitors to specifically knock down Stat3 in T cells and/or B cells in vivo or in vitro. The antibody can be a monoclonal antibody or a humanized antibody. Other delivery vehicles or means for Stat3 siRNA such as lentivirus, adenovirus, electroporation, and zinc finger nucleases can be used. In some embodiments, the therapeutic agent specifically targets Stat3 in CD4⁺ T cells and knocks down Stat3 in CD4⁺ donor or recipient T cells.

Various Stat3 siRNAs can be designed to knockdown Stat3 thereby to modulate GVHD. Some examples of the siRNA used to knock down Stat3 are shRNA targeting STAT3:

shSTAT3-1 (target sequence #1):
(SEQ ID NO: 19)
GTGTCAGATCACATGGGCTA
(antisense UAGCCCAUGUGAUCUGACAC, SEQ ID NO: 20);
shSTAT3-2 (target sequence #2):
(SEQ ID NO: 21)
GCAGCTGAACAACATGTCAT
(antisense AUGACAUGUUGUUCAGCUGC, SEQ ID NO: 22);
and
shRNA-ctrl (shRNA control sequence):
(antisense, SEQ ID NO: 23)
AGCGUUCUACACUCGACGUACU.

The term “recipient,” “host,” “subject,” or “patient” as used herein refers to a subject receiving hematopoietic cell transplantation. These terms may refer to, for example, a subject receiving an administration of donor bone marrow, donor T cells, donor spleen cells, or other donor cells or tissue. In some embodiments, the transplanted cells are derived from an allogeneic donor. The recipient, host, subject, or patient can be an animal, a mammal, or a human.

The term “donor” as used herein refers to a subject from whom the cells or tissue are obtained to be transplanted into a recipient or host. For example, a donor may be a subject from whom bone marrow, T cells, B cells, spleen cells, stem cells, or other cells or tissue to be administered to a recipient or host is derived. The donor or subject can be an animal, a mammal, or a human.

The terms “treat,” “treating,” and “treatment” as used herein with regard to a GVHD condition or an autoimmune disease refer to alleviating the condition partially or entirely, or eliminating, reducing, or slowing the development of one or more symptoms associated with the condition or disease. In some embodiments, the term “treat,” “treating,” or “treatment” means that one or more symptoms of GVHD condition, autoimmune disease, or complications are alleviated in a subject receiving the treatment as disclosed herein comparing to a subject who does not receive such treatment.

The terms “prevent,” “preventing,” and “prevention” as used herein with regard to a GVHD condition or an autoimmune disease refer to preventing the onset of the condition, disease, and/or symptoms associated with the condition or disease from occurring, decreasing the likelihood of occurrence or recurrence of the condition or disease, or slowing the progression or development of the condition or disease. For example, prevention of an autoimmune disease means preventing the onset or further development of an autoimmune disease when the subject shows signs of autoimmunity by the presence of autoantibodies in the serum but have not yet developed other symptoms of the autoimmune diseases.

The phrase “an effective amount” or “a therapeutically effective amount” as used herein refers to an amount of a therapeutic agent that produces a desired therapeutic effect. For example, an effective amount of an antibody may refer to that amount that prevents or treats GVHD or an autoimmune disease by knocking down Stat3 in the T cells and/or B cells. The precise effective amount is an amount of the therapeutic agent that will yield the most effective results in terms of efficacy in a given subject. This amount will vary depending upon a variety of factors, including but not limited to the characteristics of the therapeutic agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of the therapeutic agent and adjusting the dosage accordingly. For additional guidance, see Remington: The Science and Practice of Pharmacy (Gennaro ed. 20^{th} edition, Williams & Wilkins Pa., USA) (2000).

The term “simultaneously” as used herein with regards to administration means that the therapeutic agent or the Stat3-deficient donor T cells and/or B cells are administered to the subject at the same time or nearly at the same time of HCT. For example, the therapeutic agent or the Stat3-deficient donor T cells and/or B cells are considered to be administered “simultaneously” if they are administered via a single combined administration of hematopoietic cells, two or more administrations occurring at the same time, or two or more administrations occurring in succession without extended intervals in between.

IgG antibody production by B cells requires CD4⁺ T-cell help¹³. CD4⁺ T- and B-cell interactions occur as multistage and multifactorial processes at the extrafollicular TB border and in follicular germinal centers (GC)¹⁴. GC formation requires T- and B-cell expression of BCL6¹⁵. In brief, naive CD4⁺ T cells interact with dendritic cells (DC) in the T-cell zone of a lymphoid follicle and differentiate into Th1, Th2, Th17, and pre-Tfh under different cytokine and microenvironment regulation. Under the influence of IL-6 and ICOS signaling, CD4⁺ T cells upregulate the expression of Stat3 and BCL6, and subsequently upregulate the expression of CXCR4, CXCR5, and IL-21, downregulate the expression of CCR7 and PSGL-1(P-selectin glycoprotein ligand 1), and differentiate into pre-Tfh¹⁴. CCR7 (a ligand for CCL19 and CCL21) and PSGL-1 help anchor T cells to CCL19 and CCL21¹⁶. Downregulation of CCR7 and PSGL-1 allows the pre-Tfh cells to migrate out of the T-cell zone and reach the TB border to interact with B cells. This first stage of T-B interaction leads to the generation of short-lived plasma cells and production of low-affinity IgG1, and results in Immunoglobulin Isotype switching without somatic hypermutation¹⁷⁻¹⁹. In response to CXCL13 (a CXCR5 ligand) from follicular DCs, the CXCR5^{hi} pre-Tfh cells migrate further into the center of the B-cell zone to form Gcs^{20,21}, where the Tfh and B-cell interaction results in somatic hypermutation, production of high affinity IgG, and formation of long-lived plasma cells^{26,22}.

Extrafollicular and follicular GC CD4⁺ T- and B-cell interactions have an important function in immune defense against infections^{14,20,23}. Aberrant extrafollicular and follicular TB interactions have been observed in autoimmune diseases^{20,24,25}. For example, increased frequencies of Tfh or Tfh-like cells (CXCR5⁺PD-1^{hi} or ICOShi) are observed in the spleen of systemic autoimmune Roquinsan/san mice²⁴ and in the blood of certain patients with autoimmune Sjogren's syndrome²⁶. Mice with systemic lupus have reduced numbers of Tfh in the spleen, but the numbers of extrafollicular PSGL-1^{lo}CXCR4^{hi}CD4⁺ T cells are increased²⁵. In keeping with these observations, ectopic clusters of Tfh-like cells and B cells have been identified in the inflamed kidney tissues of patients with systemic lupus erythematosus²⁷.

Enlargement of GCs and expansion of Tfh and GC B cells have been noted in the spleens of cGVHD mice in different donor→recipient strain combinations, including C57BL/6 (H-2b)→B10. BR (H-2K), LP/J (H-2bc)→B6 and B10.D2 (H-2d)→BALB/c (H-2^{d}). Results from these studies indicate that GC formation is required for the persistence of cGVHD, suggesting that, like certain autoimmune diseases, the aberrant expansion of Tfh and B cells has an important function in cGVHD pathogenesis²⁸⁻³⁹. On the other hand, patients with active cGVHD have decreased numbers of Tfh-like cells in the blood³¹, and Tfh-like cells from the blood of cGVHD patients have an enhanced ability to stimulate IgG antibody production in vitro³². GC formation is usually associated with immunoglobulin somatic hypermutation¹⁴, but previous studies showed that allogeneic HCT recipients have gradual recovery of serum IgM, IgG1, and IgG3, but not IgG2 or IgA, and have reduced-immunoglobulin somatic hypermutation at one year after HCT, suggesting a lack of GC formation^{33,34}. Moreover, cGVHD onset is associated with lymphopenia in animal models and patients^{9-11,35}. Finally, in a cGVHD model with DBA/2 (H-2^{d}) donors and MHC-matched BALB/c recipients, it was demonstrated that although IgG antibody producing B cells and their IgG antibodies has an important function in perpetuating skin damage in cGVHD recipients, lymphoid follicles are destroyed, and GCs are not observed⁹. Taken together, these conflicting results indicate that the function of GC formation in cGVHD pathogenesis remains unclear.

After HCT with grafts containing low numbers of C57BL/6 donor splenocytes, BALB/c recipients survive acute GVHD and then develop chronic GVHD characterized by scleroderma, lymphocytic bronchitis, and damage in the salivary and lacrimal glands, which reflect features of human chronic GVHD^{7,36}. This model is used herein to explore the function of follicular and extrafollicular CD4⁺ T and B interactions in the pathogenesis of cGVHD. The results disclosed herein indicate that extrafollicular T- and B-cell interactions have important function, whereas GCs are dispensable in the pathogenesis of cGVHD.

Thus, the respective functions of extrafollicular and follicular CD4⁺ T-B interactions in cGVHD pathogenesis have remained unclear and controversial. By using different mouse models of cGVHD and donors with BCL6 deficiency specifically in B cells or CD4⁺ T cells and Stat3 deficiency in CD4⁺ T cells, the working examples demonstrate that (1) GC formation and follicular CD4⁺ T-B interaction is dispensable for induction of cGVHD, and extrafollicular CD4⁺ T- and B-cell interactions are sufficient for induction of cGVHD; (2) cGVHD pathogenesis is associated with expansion of PSGL-1^{lo}CD4⁺ T cells. PSGL-1^{lo}CD4⁺ T cells express high levels of ICOS and IL-21, and their expansion is Stat3- and BCL6-dependent; (3) BCL6 deficiency in CD4⁺ T cells prevents expansion of PSGL-1^{lo}CD4⁺ T cells and partially prevents systemic cGVHD, with effective prevention of cutaneous cGVHD; (4) Stat3 deficiency in donor CD4⁺ T cells prevents expansion of PSGL-1^{lo}CD4⁺ T cells and allows full recovery of the thymus with production of Foxp3+ Treg cells, leading to fully prevent induction of cGVHD.

As demonstrated in the working examples, GC formation is not required for induction of cGVHD. Although bone marrow transplants with BCL6 deficiency specifically in B cells (B-BCL6^{-/-}) were not able to form GCs, spleen cells from B-BCL6^{-/-} donors and WT donors induced similar severity of cGVHD. This observation is consistent with the observations that cGVHD in animal models and humans is often associated with lymphopenia at the disease onset^{9-11,35}. This observation is also consistent with the previous studies showing that allogeneic HCT patients did not have immunoglobulin somatic hypermutation, a process that requires GC formation^{33,34}. This observation is also consistent with results of a recent study showing that patients with active cGVHD had low numbers of Tfh cells in the blood³¹. However, the results disclosed herein differ from those in previous reports showing that cGVHD in murine models was associated with enlarged GCs and that GC formation was required for induction of cGVHD28-30.

Lymphopenia in cGVHD recipients results from damage to lymphoid niches¹¹. Destruction or loss of GC formation in cGVHD may also result from damage to lymphoid niches during GVHD development. Follicular DCs and lymphotoxin-producing stromal cells represent important lymphoid niches⁴⁵. Recent publications^{5,9} indicate that alloreactive and autoreactive CD4⁺ T cells and antibodies from donor B cells contribute to destruction of B-cell follicles, GCs, and follicular DCs during chronic GVHD development.

As demonstrated in the working examples, development of cGVHD is associated with expansion of PSGL-1^{lo}CD4⁺ T cells and with reduced percentages or loss of Tfh and GC B cells in the spleen. While cGVHD recipients showed loss of GCs and Tfh cells at the disease onset ˜21 days after HCT, recipients showed high percentages of PSGL-1^{lo}CD4⁺ T cells in the spleen, liver, and lung. The percentages of PSGL-1^{lo}CD4⁺ T cells declined to control levels at the peak time of cGVHD, ˜45 days after HCT. PSGL-1^{lo}CD4⁺ T cells expressed high levels of ICOS, IL-21, IL-13, and IL-17. Blockade of ICOS signaling effectively prevented their expansion and effectively ameliorated cGVHD. In addition, add-back of PSGL^{lo}CD4⁺ T cells to recipients given CD4-BCL6^{-/-} transplants that could not give rise to PSGL1^{lo}CD4⁺ T cells resulted in marked augmentation of damage in the thymus and development of cutaneous cGVHD. These results indicate that PSGL^{lo}CD4⁺ T cells are important in the pathogenesis of cGVHD.

Disclosed herein is the role of ICOS/ICOS-ligand interaction in augmenting expansion of extrafollicular PSGL-1^{lo}CD4⁺ T cells. Since there was an absence of GC in cGVHD recipients, amelioration of cGVHD by blockade of ICOS/ICOS-ligand interaction described in a previous publication²⁸ may not result from reduction of GC formation, but may result from blockade of interactions between extrafollicular CD4⁺ T cells and B cells. This is supported by the observations that development of lupus in MRL^{lpr} mice is associated with expansion of PSGL-1^{lo}CD4⁺ T cells but not with expansion of Tfh cells²⁵.

It was reported that CD4⁺ T and B interactions and antibody production are required to perpetuate cGVHD and augment Th17 infiltration of skin tissues^{8,9}, suggesting that CD4⁺ T- and B-cell interaction may continue in GVHD target tissues, even after destruction of lymphatic tissues. As demonstrated in the working examples, the percentages of PSGL-1^{lo}CD4⁺ T cells in the spleen decreased shortly after the onset of cGVHD but persisted much longer in GVHD target tissues. Local T/B interactions in inflamed tissues is supported by non-Tfh CXCR5-CD4⁺ helper T cells^{49,50}. PSGL-1^{lo}CD4⁺ T cells and B cells may interact in GVHD target tissues in the pathogenesis of cGVHD.

Early after activation, CD4⁺ T cells downregulate CCR7 and PSGL-1 and migrate to T-B border; some of the CD4⁺ T cells become pre-Tfh under influence of cytokines (such as IL-6 and IL-21), with upregulation of Stat3 and BCL6 as well as CXCR5 and ICOS. Pre-Tfh CD4⁺ T cells can interact with B cells at the T-B border to produce IgG antibodies^{14,28}. As demonstrated in the working examples, like pre-Tfh cells, PSGL-1^{lo}CD4⁺ T cells in cGVHD are also Stat3 and BCL6 dependent and express high levels of ICOS and IL-21. Therefore, PSGL-1^{lo}CD4⁺ T cells can develop at the TB border during the pre-Tfh stage of development. Unlike pre-Tfh cells, however, PSGL-1^{lo}CD4⁺ T cells in cGVHD recipients express low levels of CXCR5. PSGL-1^{lo}CD4⁺ T cells in cGVHD recipients could be a subset of pre-Tfh cells because pre-Tfh cells are heterogeneous, and some of them are CXCR5^{lo28}.

It was reported that high levels of IL-6 are associated with onset of cGVHD^{35,51,52}. GVHD-related alloimmunity damages the host-type follicular DC and stromal cells in lymphoid tissues early after HCT, before donor-type follicular DC and stromal cells join the lymphoid structure. Donor-type CD4⁺ T and B cells can interact at TB border but cannot move forward to form intact lymphoid follicles or GCs.

IgG antibody production in cGVHD recipients is associated with destruction of lymphoid tissues⁹, suggesting that IgG antibodies resulting from extrafollicular CD4⁺ T and B interactions may augment damage to lymphoid structures, which can also prevent GC formation. Recipients given CD4-BCL6^{-/-} CD4⁺ T cells had lymphoid follicles but did not have GCs, unlike the destruction of lymphoid follicles in recipients given WT CD4⁺ T cells with cGVHD. This indicates that the interaction of autoreactive PSGL-1^{lo}CD4⁺ T cells with B cells and the production of IgG autoantibodies are important in damaging lymphoid structures.

Stat3 deficiency in donor CD4⁺ T cells fully prevented induction of cGVHD, but BCL6 deficiency in donor CD4⁺ T cells only partially prevented cGVHD, although both effectively prevented expansion of PSGL-1^{lo}CD4⁺ T cells. The difference may result from the ability of Stat3 to regulate development of Foxp3⁺ Treg and RoRγt⁺ Th17 cells^{53,54} in addition to BCL6⁺ pre-Tfh and Tfh cells¹⁴, whereas BCL6 in CD4⁺ T cells regulates only the development of pre-Tfh and Tfh^{39,55}. Stat3 deficiency in donor CD4⁺ T cells not only reduced expansion of PSGL-1^{lo}CD4⁺ T cells and reduced their infiltration of thymus, but also reduced serum concentrations of anti-dsDNA autoantibody and deposition in thymus tissues, allowing for full recovery of the thymus and its production of Treg cells that control peripheral autoreactive T-cell expansion, leading to full prevention of cGVHD development. Thus, Stat3 deficiency in donor CD4⁺ T cells allows full recovery of the thymus and production of natural Treg cells.

Infusion or in vivo expansion of donor-type Treg cells can ameliorate ongoing cGVHD^{57,58}. Deletion of Stat3 and BCL6 in CD4⁺ T cells decreases the numbers of follicular regulatory Treg cells^{59,60}. As demonstrated herein, GC were tiny or absent in recipients given TCDBM only or TCD-BM and spleen cells from donors with specific deletion of Stat3 or BCL6 in CD4⁺ T cells, suggesting that those recipients have low numbers of follicular Treg cells. However, cGVHD was absent in recipients given Stat3-deficient CD4⁺ T cells and showed decreased severity in recipients given BCL6-deficient CD4⁺ T cells as compared to recipients given wild-type CD4⁺ T cells. Extrafollicular CD4⁺ T and B interaction is important in induction of cGVHD, and that peripheral Treg cells but not follicular Treg cells can regulate cGVHD development.

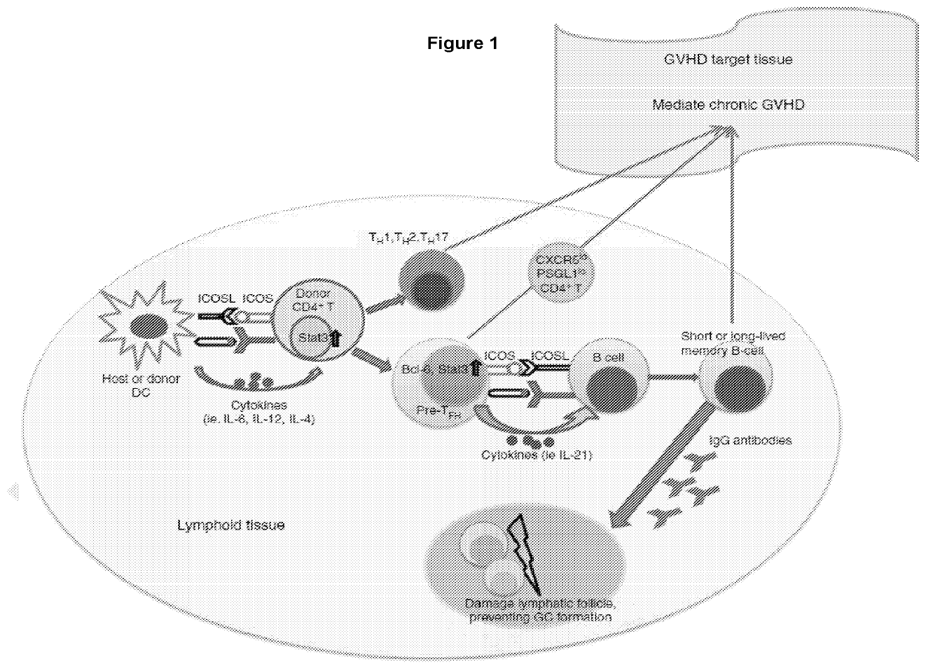

The pathogenesis of chronic GVHD is a complex process involving different Th subsets (i.e., Th1, Th2, and Th17), pre-Tfh like PSGL-1^{lo}CD4⁺ T cells, B cells, and autoantibodies. The inventors discovered the important function of pre-Tfh-like PSGL-1^{lo}CD4⁺ T cells and their interaction with B cells in the pathogenesis of this disease. As depicted FIG. 1, during cGVHD development, allo- and autoreactive CD4⁺ T cells may first interact with host or donor-type DCs and differentiate into various Th subsets (i.e., Th1, Th2, and Th17), and some CD4⁺ T cells differentiate into pre-Tfh cells under the influence of IL-6 and ICOS. The pre-Tfh cells upregulate expression of Stat3, BCL6, IL-21, and CXCR4, but downregulate CCR7 and PSGL-1, and migrate from the T-cell zone to the TB border. Unlike pre-Tfh cells in the lymphoid tissues of healthy mice that upregulate CXCR5 and migrate into the center of B-cell zone, the pre-Tfh-like PSGL-1^{lo}CD4⁺ T cells in cGVHD recipients maintain low-level expression of CXCR5 and persist at the TB border, where they interact with donor B cells to produce low-affinity IgG1 with no somatic hypermutation. Pathogenic PSGL-1^{lo}CD4⁺ T cells and deposition of IgG1 antibodies damage lymphoid follicles, thereby preventing GC formation. In addition, pre-Tfh-like PSGL-1^{lo} CD4⁺ T cells and B cells may migrate into GVHD target tissues, such as skin and lung, to interact and produce IgG antibodies that augment the pathogenesis of cGVHD, which is exacerbated by infiltration of Th1 and Th17 cells^{6,9}. CXCR5-PD-1^{hi} non-Tfh but Tfh-like CD4⁺ T cells interact with B cells in the inflamed tissues in mice with experimental allergic pneumonia⁴⁹ and in humans with rheumatoid arthritis⁵⁰. Thus, blocking the expansion of pre-Tfh-like PSGL-1^{lo}CD4⁺ T cells and augmenting the expansion of Foxp3⁺ Treg cells by blocking or targeting Stat3 in CD4⁺ T cells may be an effective approach for preventing or treating cGVHD.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Materials and Methods

Mice.

BALB/c (H-2^{d}) and C57BL/6(H-2^{b}) mice were purchased from National Cancer Institute Laboratories (Frederick, Md.). LP/J (H-2^{b}), B10.D2 (H-2^{d}), B10. BR (H-2^{k}) mice were purchased from The Jackson Laboratory (Bar Harbor, Me.). BCL6^{fl/fl} Mb1-Cre C57BL/6 was obtained from Dr Markus Muschen's lab at University of California San Francisco (San Francisco, Calif.)⁴⁰. Stat3^{fl/fl}CD4-Cre C57BL/6 was obtained from Dr Hua Yu's lab at City of Hope National Medical Centre (Duarte, Calif.)⁶¹. BCL6^{fl/fl} C57BL/6 was mated with CD4-Cre-057BL/6 to generate BCL6^{l/fl} CD4-Cre-057BL/6 mice, and mice over fifth generations were used for experiment. Mice were maintained in a specific pathogen-free room at the City of Hope Research Animal Facilities (Duarte, Calif.). Eight to 12 weeks old male mice were used for all experiments. Mice were killed using CO₂. All animal protocols were approved by the City of Hope Institutional Animal Care and Use Committee.

Induction and Assessment of GVHD.

The mice were irradiated by using a ¹³⁷Cs source at a dose of 850 cGy 8-10 h before HCT. Recipients were injected with 2.5×10⁶ T-cell-depleted donor BM cells (TCD-BM) alone or 2.5×10⁶ TCD-BM together with splenocytes 0.01×10⁶ or 1×10⁶ from C57BL/6 donors, 0.25 or 1×10⁶ from B10.BR donors, or 10×10⁶ from LP/J or B10.D2 donors.

The assessment and scoring of clinical cutaneous GVHD were performed according to previous publications with mild modification^{7,9}. Mice were evaluated and scored based on the development of alopecia and ulcers on hair-bearing skin. Ulcers or scaling in non-hair-bearing skin (ears, tails, and paws) were also examined for scoring. (1) skin ulcers with alopecia <1 cm² in area; (2) skin ulcers with alopecia 1-2 cm² in area; (3) skin ulcers with alopecia >2 cm²; (4) skin ulcers with alopecia >30% body area.

Histopathology.

Hematoxylin and eosin (HE) staining on formalin fixed paraffin embedded tissue slides were used for evaluation. Slides were examined at ×200 magnification, and tissue damage was blindly assessed according to the previous publications^{7,9}. Salivary gland GVHD was evaluated based on mononuclear cell infiltration and structural disruption, with a maximum score of 8. Skin GVHD was scored according to damage in the epidermis and dermis as judged by hyperplasia of epidermis, enlargement, fibrosis of dermis, and loss of subcutaneous fat with the maximum score⁹. Lung tissue was evaluated on a scoring system based on perivascular and peribronchiolar infiltration and inflammation; the maximum score is 9. Liver was scored on the number of involved tracts and the severity of lymphocytic infiltration and liver cell necrosis with the maximum score of 9.

Antibodies for Histoimmunofluorescent Staining.

FITC-labeled anti-mouse CD3 (145-2C11, #11-0031-82, 1:100) and GL7 (GL7, #13-5902-81, 1:200) were purchased from eBiosciences (San Diego, Calif.). Rat anti-mouse B220 (RA3-6B2, #550286, 1:100) and rat anti-mouse IgD (11-26c.2a, #553438, 1:100) were purchased from BD Biosciences (San Jose, USA). Biotinylated PNA (# B-1075, 1:300), goat anti-mouse IgM (# FI-2020, 1:100) and AMCA-labeled streptavidin (# SA-5008, 1:300), biotinylated ulex europaeus agglutinin-1 (UEA-1, #61065, 1:200) and FITC-labeled streptavidin (# SA-5001, 1:100) were purchased from Vector Laboratories (Burlingame, USA). Texas red-labeled goat anti-rat IgG (# T-6392, 1:500), Alexa Fluor® 488-labeled goat anti-rat IgG (# A-11006, 1:500), and Alexa Fluor® 488-labeled goat anti-mouse IgG (# A11001, 1:200) were purchased from Thermo Fisher Scientific (Waltham, USA). Rat anti-mouse cytokeratin 8 (# Troma-1 1:100) was purchased from Developmental Studies Hybridoma Bank (Iowa City, USA).

GC detection. Spleen was embedded in optimum cutting temperature compound and 5 μm thickness cryosections were used for staining. Three staining combinations were performed to detect GCs according to previous publications in order to ensure the reliability of staining. Combination 1: cryosections were stained with FITC-labeled anti-mouse CD3 combined with rat anti-mouse B220 and biotinylated PNA, followed by Texas red-labeled goat anti-rat IgG secondary antibody and AMCA-labeled streptavidin. Combination 2: cryosections were stained with FITC-labeled goat anti-mouse IgM and biotinylated PNA followed by AMCAstreptavidin. Combination 3: cryosections were stained with rat anti-mouse IgD and biotinylated GL7 followed with Alexa Fluor® 488-labeled goat anti-rat IgG secondary antibody and AMCA-streptavidin. Images were acquired on Olympus BX50 immunofluorescent microscope (Olympus, Center Valley, USA) at ×200 magnification.

IgG Deposition and Thymic Epithelial Cells Staining.

The detection of IgG deposition in skin and thymus tissues, and thymus epithelial cell staining was performed according to previous publications of our group and others with modest modification^{9,43}. Skin and thymus cryosections were stained with Alexa Fluor® 488-labeled goat anti-mouse IgG for IgG deposition. Thymus cryosections were stained with biotinylated ulex europaeus agglutinin-1 (UEA-1) for medullary epithelial cells, and rat anti-mouse cytokeratin 8 for cortical epithelial cells followed by FITC-streptavidin or Texas red-labeled goat anti-rat IgG. Images were acquired on Olympus BX50 immunofluorescent microscope (Olympus, Center Valley, USA) at ×200 magnification.

Real-time quantitative PCR. RNA samples were isolated from sorted CD4⁺CD44^{hi}PSGL-1^{lo}CD62L^{lo} T cells by using miRNeasy Mini Kit purchased from Qiagen (Valencia, Calif., #217004). Real-time qualitative PCR were performed using SYBR Green Supermix (Bio-Rad, Hercules, Calif. #172-5124) after RNA was reverse transcribed into cDNA with reverse transcription kits (Bio-Rad, Hercules, Calif., #170-8890). Relative gene expression was normalized to GAPDH. The primers used for CCR7, CXCR4, CXCR5, BCL6, STAT3, ICOS, PDCD-1, CD80, PD-L1, and GAPDH amplification have been described in previous publications by our group and others⁶²⁻⁶⁹.

The sequences of the primers were as follows:

TABLE 1

| Genes | Primers | Sequences |
|---|---|---|
| CCR7 | Forward | 5′-TGGTGGCTCTCCTTGTCATTT-3′ |
| | | (SEQ ID NO: 1) |
| | Reverse | 5′-ACCGACTCGTACAGGGTGTAGTC-3′ |
| | | (SEQ ID NO: 2) |
| CXCR4 | Forward | 5′-CATGGAACCGATCAGTGTGAGT-3′ |
| | | (SEQ ID NO: 3) |
| | Reverse | 5′-GCAGGGTTCCTTGTTGGAGT-3′ |
| | | (SEQ ID NO: 4) |
| CXCR5 | Forward | 5′-ACTCCTTACCACAGTGCACCTT-3′ |
| | | (SEQ ID NO: 5) |
| | Reverse | 5′-GGAAACGGGAGGTGAACCA-3′ |
| | | (SEQ ID NO: 6) |
| BCL6 | Forward | 5′-CACACCCGTCCATCATTGAA-3′ |
| | | (SEQ ID NO: 7) |
| | Reverse | 5′-TGTCCTCACGGTGCCTTTTT-3′ |
| | | (SEQ ID NO: 8) |
| ICOS | Forward | 5′-CTCACCAAGACCAAGGGAAGC-3′ |
| | | (SEQ ID NO: 9) |
| | Reverse | 5′-CCACAACGAAAGCTGCACACC-3′ |
| | | (SEQ ID NO: 10) |
| PDCD-1 | Forward | 5′-CGTCCCTCAGTCAAGAGGAG-3′ |
| | | (SEQ ID NO: 11) |
| | Reverse | 5′-GTCCCTAGAAGTGCCCAACA-3′ |
| | | (SEQ ID NO: 12) |
| CD80 | Forward | 5′-GGCAAGGCAGCAATACCTTA-3′ |
| | | (SEQ ID NO: 13) |
| | Reverse | 5′-CTCTTTGTGCTGCTGATTCG-3′ |
| | | (SEQ ID NO: 14) |
| PD-L1 | Forward | 5′-AGGATATTTGCTGGCATTATATTCAC-3′ |
| | | (SEQ ID NO: 15) |
| | Reverse | 5′-ACAAACTGAATCACTTGCTCATCTT-3′ |
| | | (SEQ ID NO: 16) |
| GAPDH | Forward | 5′-TCACCACCATGGAGAAGGC-3′ |
| | | (SEQ ID NO: 17) |
| | Reverse | 5′-GCTAAGCAGTTGGTGGTGCA-3′ |
| | | (SEQ ID NO: 18) |

RNA Sequencing Analysis.

RNA samples were isolated from sorted CD4⁺CD44^{hi}PSGL-1^{lo}CD62L^{lo} T cells by using miRNeasy Mini Kit purchased from Qiagen (Valencia, USA, #217004). Total RNA sequencing was performed and analyzed by the Integrative Genomics Core, City of Hope National Medical Center (Duarte, Calif.). Transcriptome libraries were constructed with TruSeq Stranded Total RNA Ribo-Zero Kit (IIlumina, CA, # RS-122-2203). In brief, 500 ng of total RNA from each sample was used to construct a cDNA library, followed by sequencing on the Illumina Hiseq 2500 with single end 50 bp reads according to the manufacturer's recommendations. The sequences were aligned to mouse genome assembly mm9 using Tophat2. For each sample, total counts for Refseq genes were summarized by HTseq⁷⁶ and reads per kilobase of transcript per million mapped reads (RPKM) were calculated. The RNA expression levels of the genes of interest were shown as mean centered log₂ RPKM by heat maps generated by Java Treeview. Counts were normalized, and differential expression analysis between groups was conducted by using the Bioconductor package “edgeR.”

Flow Cytometry Analysis and Cell Sorting.

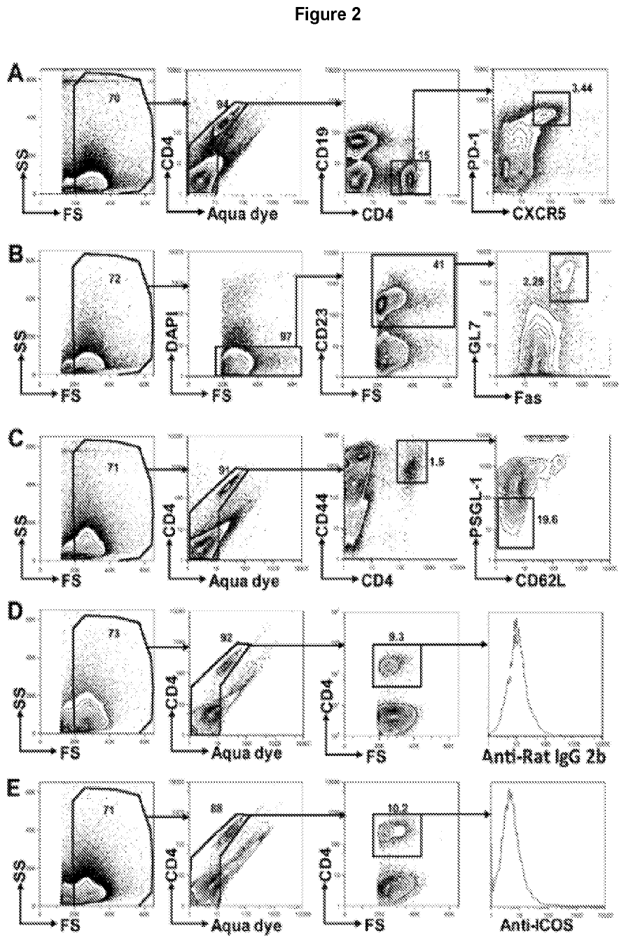

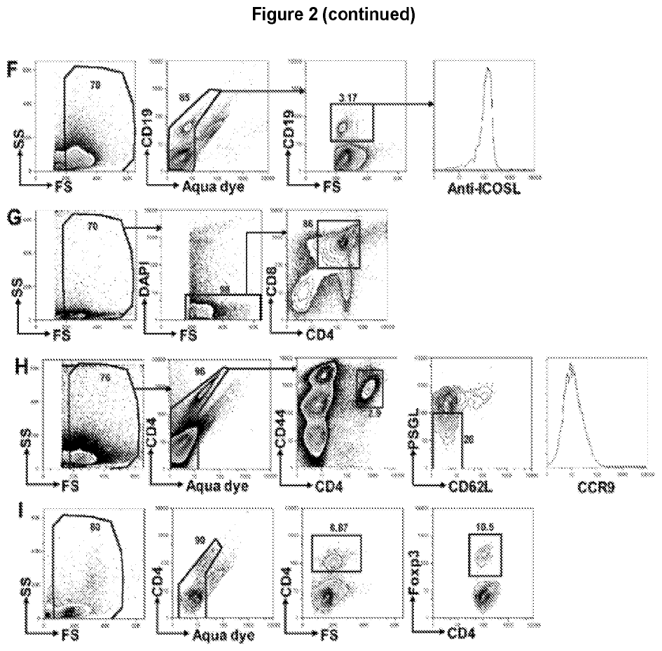

The antibodies and reagents used for flow cytometry analysis and cell sorting are listed as below: antibodies to mouse, CD4 (RM4-5, #48-0042-82, 1:100), CD44 (IM7, #25-0441-81, 1:300), CD62L (MEL-14, #11-0621-85, 1:100), CD19 (eBio 1D3, #11-0193-81, 1:100), GL7 (GL7, #13-5902-81, 1:100), PD-1 (J43, #17-9985-82, 1:200), CD23 (B3B4, #13-0232-81, 1:200), IgM (II/41, #17-5790-82, 1:200), IgD (11-26c, #12-5993-82, 1:300), interleukin 21 (FFA21, #12-7211-80, 1:100), interleukin-13 (eBio13A, #12-7133-82, 1:100), interferon-γ (XMG1.2, #12-7311-82, 1:100), interleukin-17 (eBio 1767, #12-7177-81, 1:100), ICOS (CD278, 7E.17G9, #12-9942-81, 1:200), BCL-6 (BCL-DWN, #12-5453-80, 1:100), FasL (CD178, MFL3, #13-5911-82, 1:100), and rat IgG 2a isotype control antibody (eBR2a, #12-4321-81, 1:100), anti-rat IgG2b (R26-7C3, #13-4815-80, 1:200), streptavidin (17-4317-82, 1:300) were all purchased from eBiosciences (San Diego, Calif.). Anti-mouse ICOSL (CD275, HK5.3, #107403, 1:100) was purchased from Biolegend (San Diego, USA). Fas (Jo2, #554258, 1:200), CD21 (7G6, #561769, 1:100), PSGL-1 (2PH1, #562806 1:300) and CXCR5 (2G8, #551960, 1:200) were purchased from BD Biosciences (San Jose, USA). Antibody to mouse CCR9 (242503, # FAB2160P, 10 μl/10⁶ cell) was purchased from R&D (Minneapolis, USA). FoxP3 staining kit (00-5523-00) was purchased from eBioscience (San Diego, Calif.). Aqua fluorescent reactive dye for viability analysis (L34957) was obtained from Invitrogen (Carlslad, Calif.). All staining was performed according to the manufacturer's instructions. Flow cytometric analysis was performed on CyAn Immunocytometry system (DAKO Cytomation, Fort Collins, Colo.) and data were analyzed with FlowJo software (Tree Star, Ashland, Oreg.). Extrafollicular PSGL-1^{lo}CD4⁺ cells were sorted with a 6-laser Arial III immunocytometry system (DakoCytomation, Fort Collins, USA) and used for intracellular cytokine staining, RNA-seq analysis and in vivo adoptive transfer experiments. The gating strategies were shown in FIG. 2.

In Vivo Anti-ICOS Treatment.

Anti-mouse ICOS (7E.17G9) was purchased from Bio-X cell (West Lebanon, USA, # BE0059). The in vivo anti-ICOS treatment was performed according to previous report with modification²⁸. The treatment started on the day of transplantation, and recipients were injected i.p. with 200 μg/mouse anti-mouse ICOS or isotype control (rat IgG2b, # BE0090) every other day until 45 days after transplantation.

Statistical Analysis.

Survival in different groups was compared using the log-rank test with the program GraphPad Prism, version 6.0 (GraphPad Software, San Diego, Calif.). Two-way ANOVA was used for comparing body weight changes and cutaneous cGVHD score in different groups. Unpaired two-tailed Student's t test was used to determine significant differences between two experimental groups.

Data Availability.

The total RNA sequencing data have been deposited and are available in the GEO database (GSE101552).

### Example 1: Onset of cGVHD and Lymphopenia is Associated with the Absence of GCs

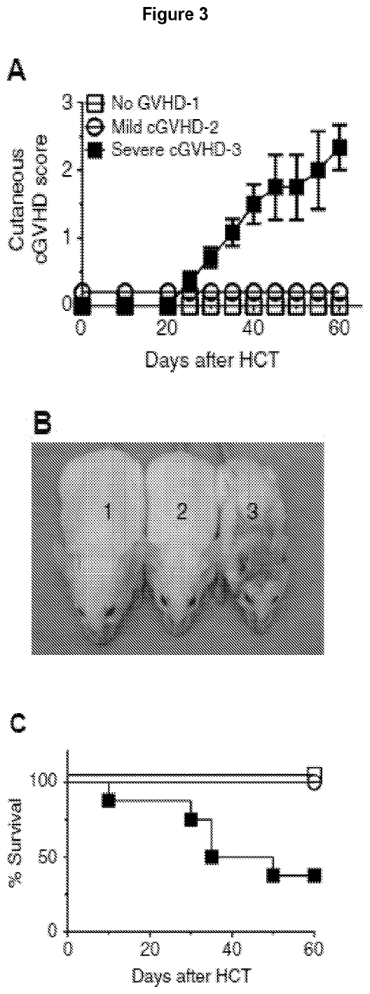

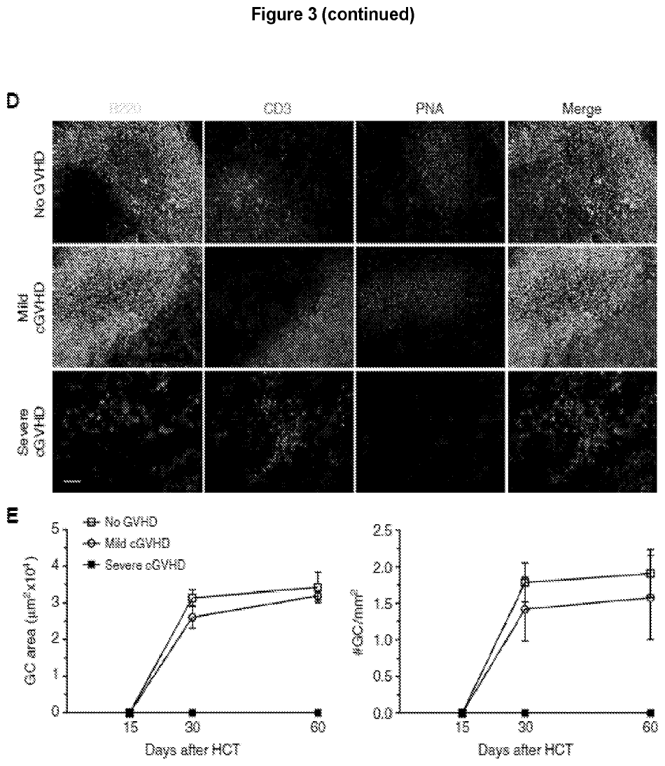

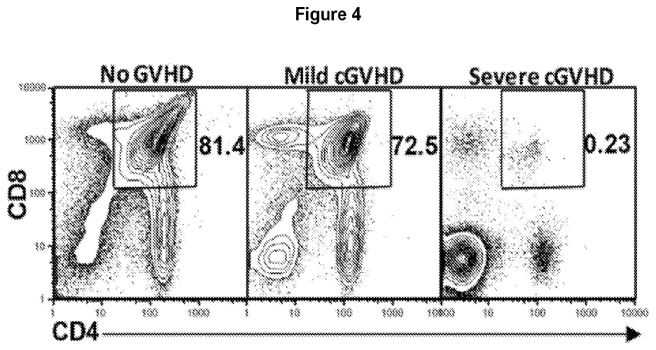

To address the role of GC formation in cGVHD pathogenesis, BALB/c recipients were injected with T-cell depleted bone marrow (TCD-BM) plus 1×10⁶ or 0.01×10⁶ spleen cells from C57BL/6 donors. Recipients given TCD-BM alone showed no signs of GVHD (no-cGVHD). Recipients given 1×10⁶ spleen cells developed severe clinical cGVHD with hair loss, and only ˜40% survived for more than 60 days (severe cGVHD) (FIGS. 3A-3C). Recipients given 0.01×10⁶ spleen cells showed mild signs of cGVHD (FIGS. 3A-3C) and mild reduction of thymic CD4⁺CD8⁺ thymocytes (FIG. 4).

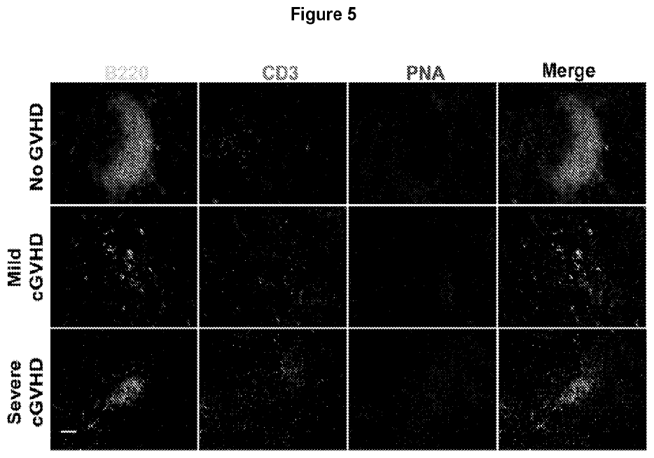

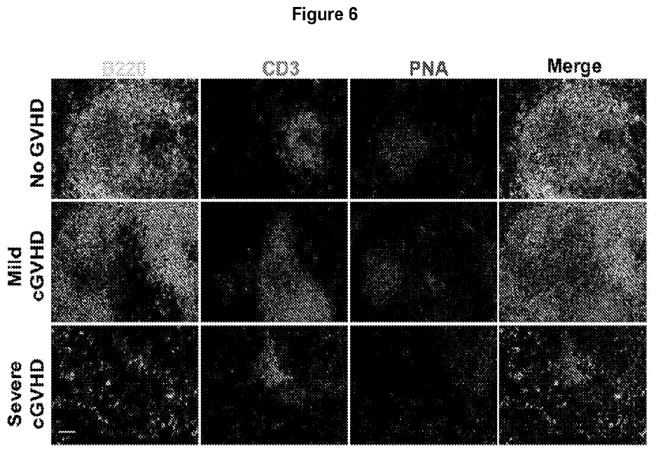

Lymphoid follicles and GCs in the spleen of the recipients were measured with immunofluorescent staining of tissue sections. Anti-B220 was used to visualize B-cell zones, anti-CD3 to visualize T-cell zones and peanut agglutinin (PNA) to identify GCs (FIG. 3D). The GC areas and numbers were measured at days 15, 30, and 60 after HCT. No GCs were found at 15 days after HCT in any recipients (FIG. 5). Well-formed GCs were present in no-GVHD recipients at 30 and 60 days after HCT (FIGS. 6 and 3D). Recipients with mild cGVHD also had GC formation, slightly reduced in size and numbers, as compared to no-GVHD recipients (FIGS. 3D, 3E and 6). GC enlargement was not observed in any recipients at any time. In contrast, no GCs were observed at any time in recipients with severe cGVHD (FIGS. 3D, 5 and 6).

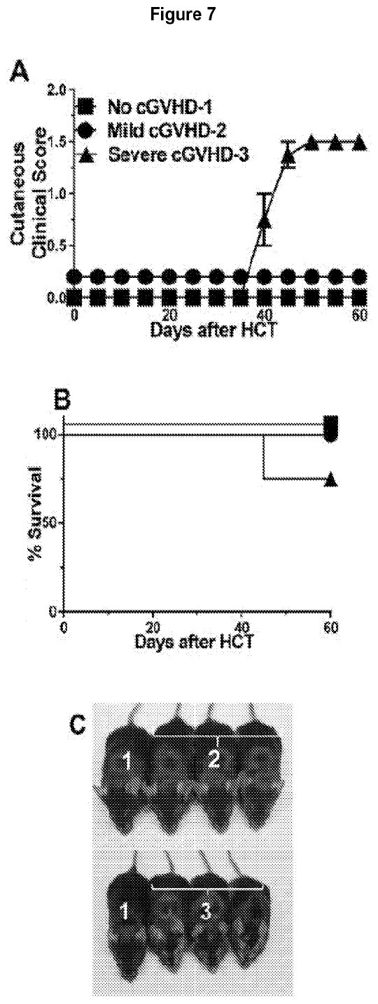

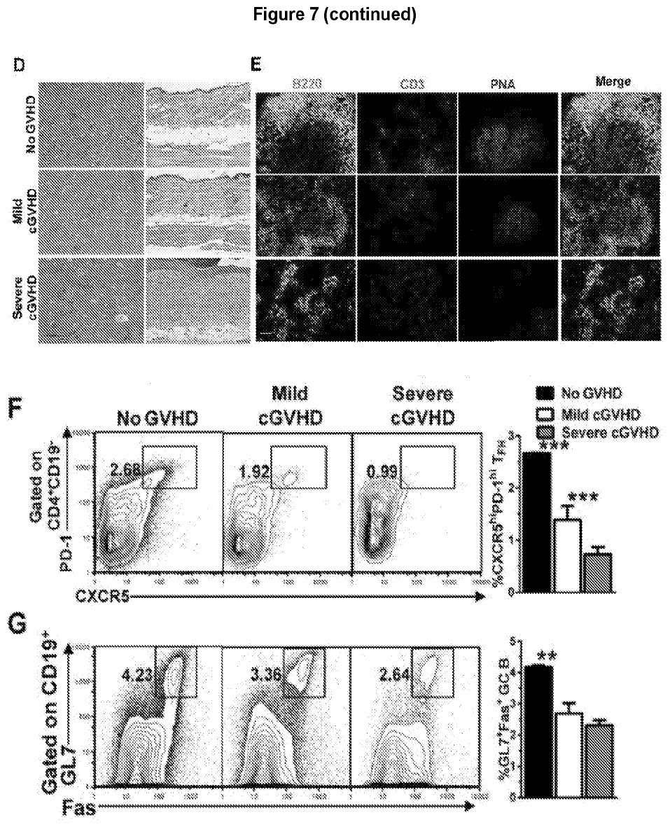

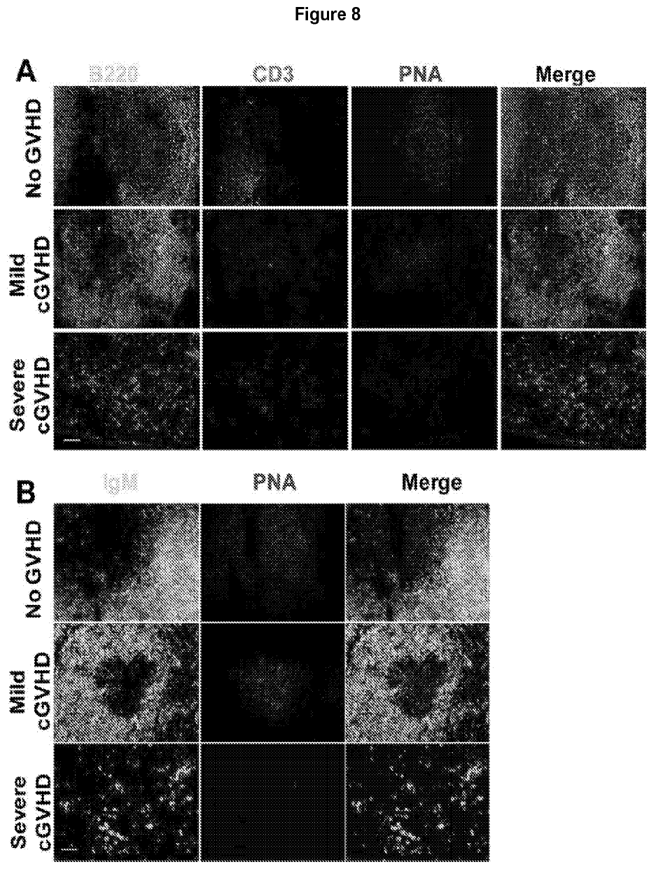

To further validate these initial observations, additional experiments were carried out in three other donor-recipient strain combinations²⁸⁻³⁰. In the MHC-matched LP/J*C57BL/6 model, recipients were divided into two groups, severe cGVHD and mild cGVHD, based on the severity of cutaneous cGVHD, survival, and histopathology (FIGS. 7A-7D). GC formation in the spleen was measured at 60 days after HCT. Compared to no-GVHD recipients given TCD-BM alone, recipients with severe cGVHD showed destruction of lymphoid follicles and absence of GCs (FIG. 7E). Recipients with mild cGVHD had lymphoid follicles and GC formation, but the GCs appeared to be smaller than in no-GVHD recipients (FIG. 7E). These results were consistent with flow cytometry showing decreased percentages of CXCR5^{hi}PD-1^{hi} T_{FH} and Fas⁺GL7⁺ GC B cells in splenocytes from recipients with cGVHD (FIGS. 7F, 7G). Results of immunofluorescent tissue staining were similar in experiments with the C57BL/6→B10.BR model (FIG. 8A).

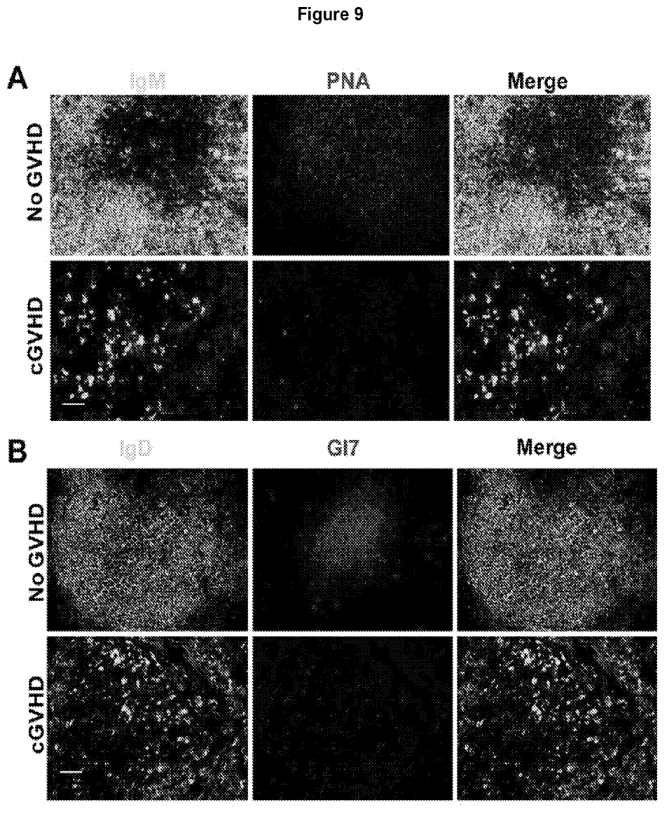

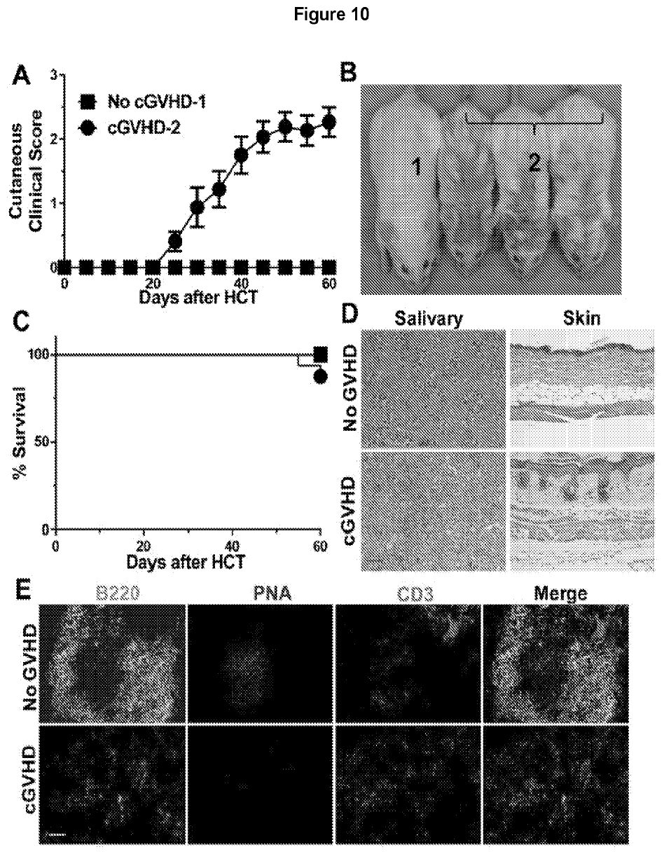

To further validate the staining of GCs, the combination of IgM and PNA or IgD and GL7 was used to visualize GCs, as performed by others^{30,37}. In the C57B/6→B10.BR model, smaller GCs were observed in recipients with mild cGVHD, and no GCs were observed in the recipients with severe cGVHD (FIG. 8B). Additionally, in LP/J→C57BL/6 model, no-GVHD recipients given TCD-BM alone showed lymphoid follicles and GCs, but cGVHD recipients showed destruction of lymphoid follicles and absence of GCs under two different staining combinations (FIGS. 9A, 9B). Chronic GVHD was also associated with destruction of lymphoid follicles and absence of GCs in the MHC-matched B10.D2→BALB/c model³⁸ (FIG. 10). Taken together, these results show that cGVHD is associated with destruction of lymphoid follicles and absence of GCs.

### Example 2: Chronic GVHD is Induced in the Absence of GC Formation

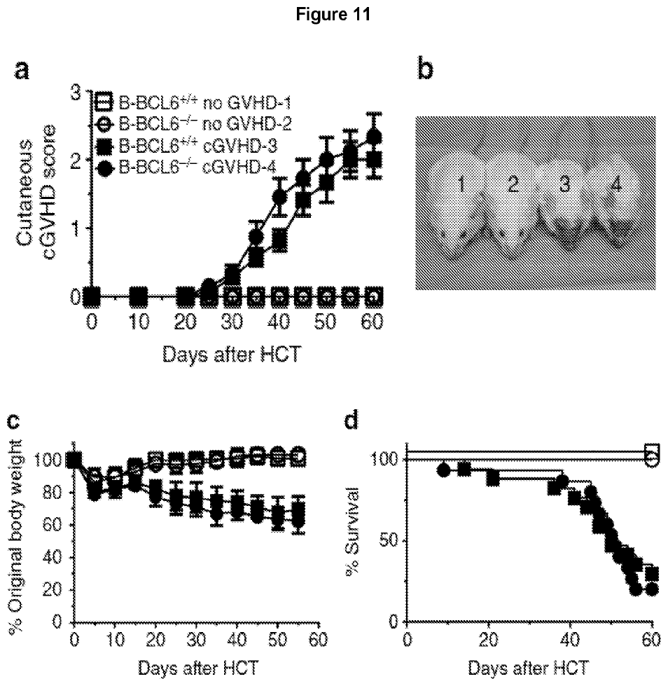

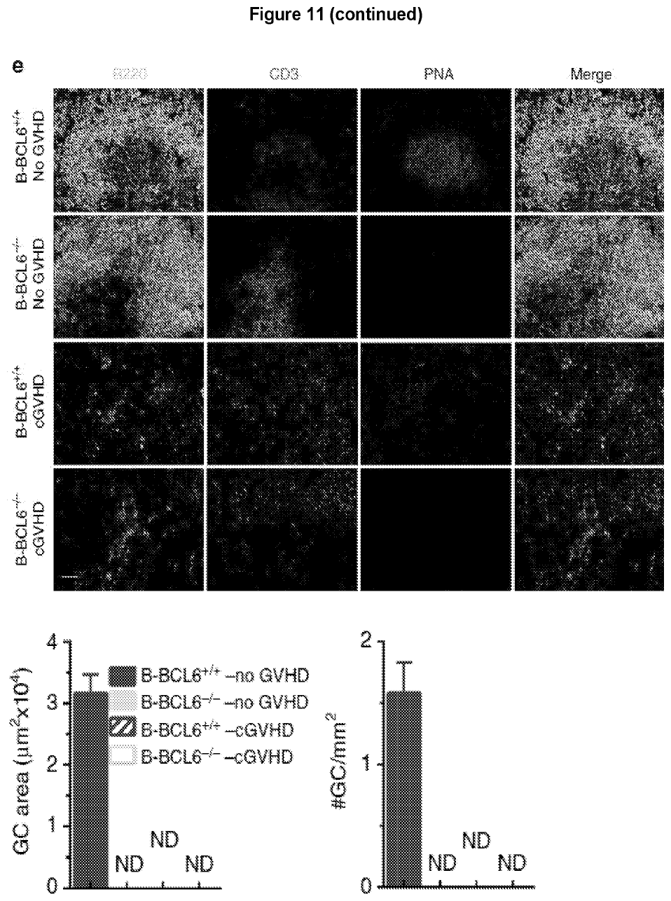

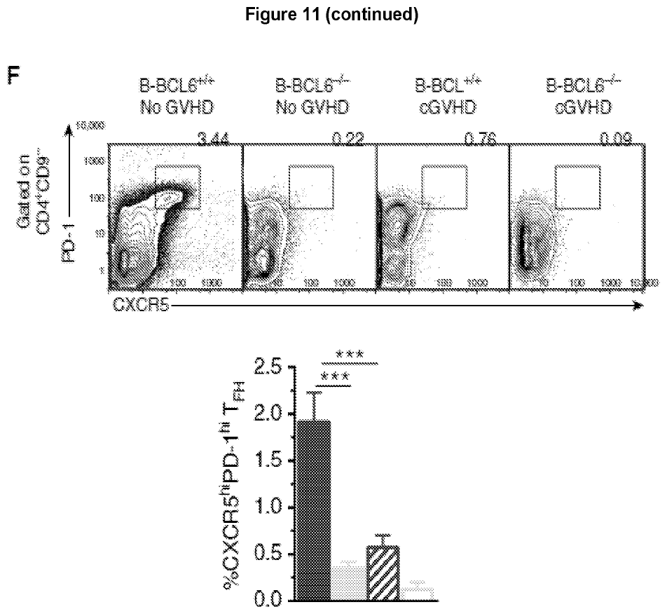

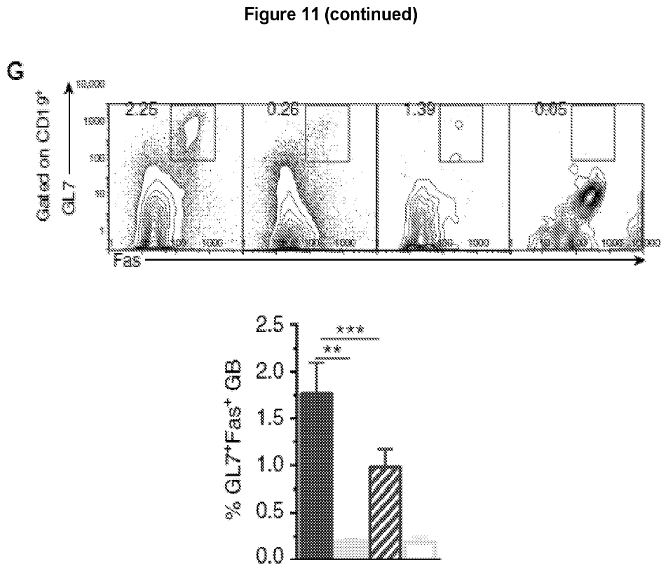

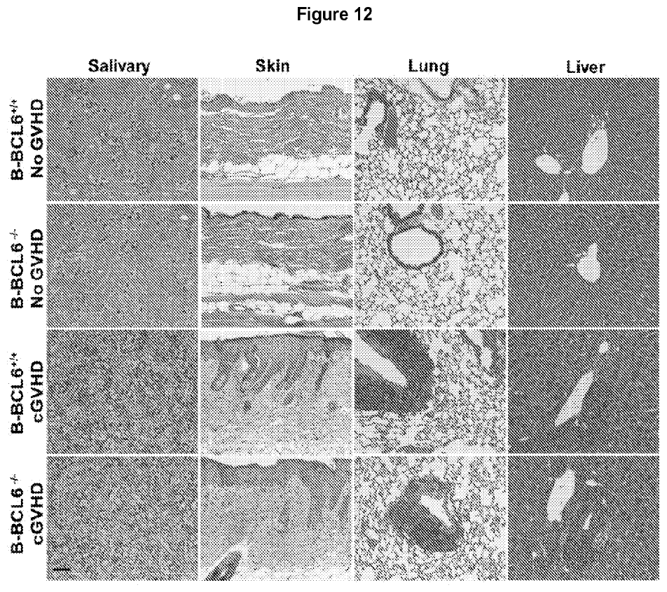

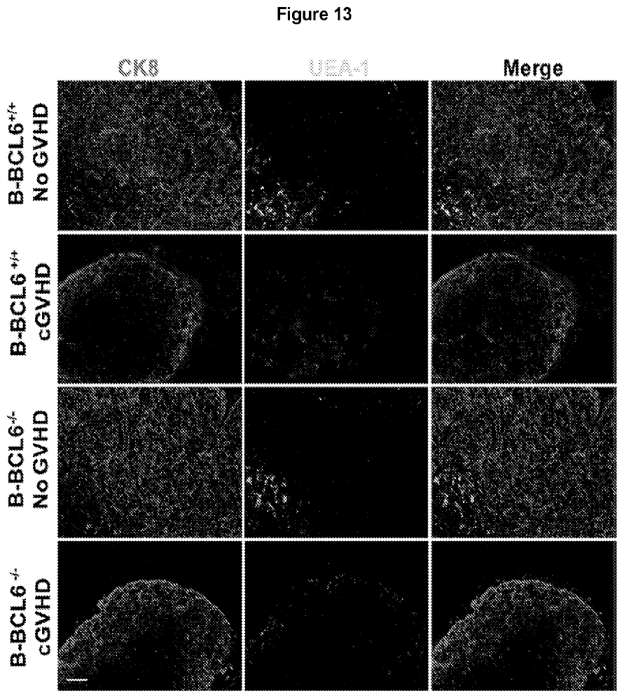

Whether induction of cGVHD requires GC formation was tested. Mice with BCL6 deficiency in T or B cells cannot form GCs15, 39. BCL6^{fl/fl}Mb1-Cre^{+/-}C57 BL/6 mice have BCL6 deficiency specifically in B cells (B-BCL6^{-/-})⁴⁰. TCD-BM and spleen cells from B-BCL6^{-/-} or B-BCL6^{+/+} control littermates in the C57BL/6 background were transplanted into lethally irradiated BALB/c recipients as described in Materials and Methods section above and a previous publication⁷. Recipients given B-BCL6^{+/+} or B-BCL6^{-/-} TCD-BM cells (2.5×10⁶) alone showed no signs of GVHD (B-BCL6^{+/+} or B-BCL6^{-/-} no GVHD). Recipients given additional spleen cells (1×10⁶) from B-BCL6^{+/+} or B-BCL6^{-/-} mice developed severe cutaneous cGVHD with hair loss (B-BCL6^{+/+}- or B-BCL6^{-/-}-cGVHD), and ˜60% died by 60 days after HCT. No significant differences were found between the two groups, as judged by clinical manifestations (FIGS. 11A-11D), histopathology (FIG. 12), or thymus damage (FIG. 13).

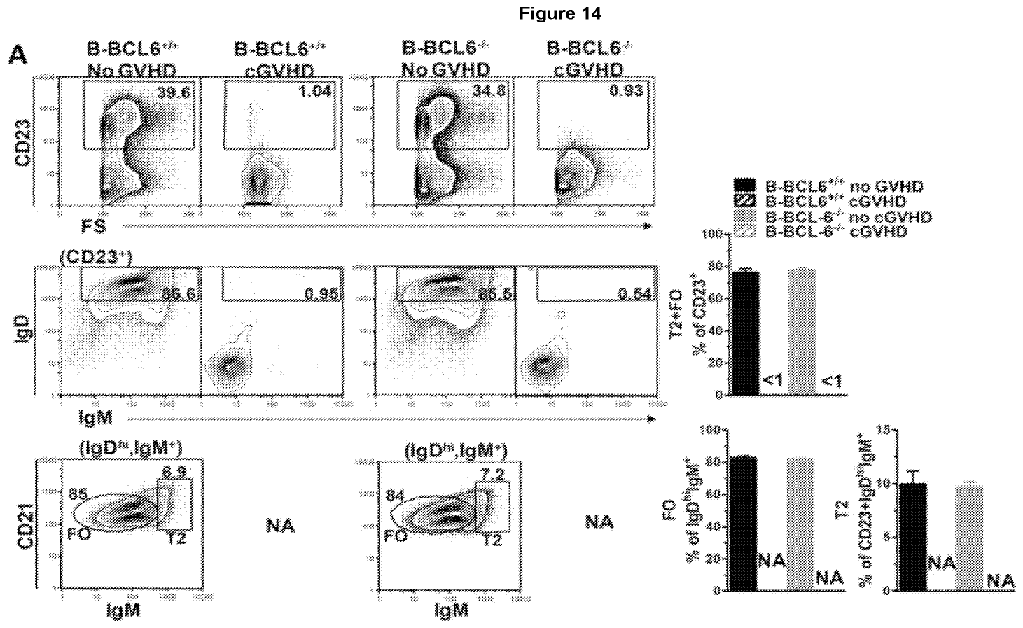

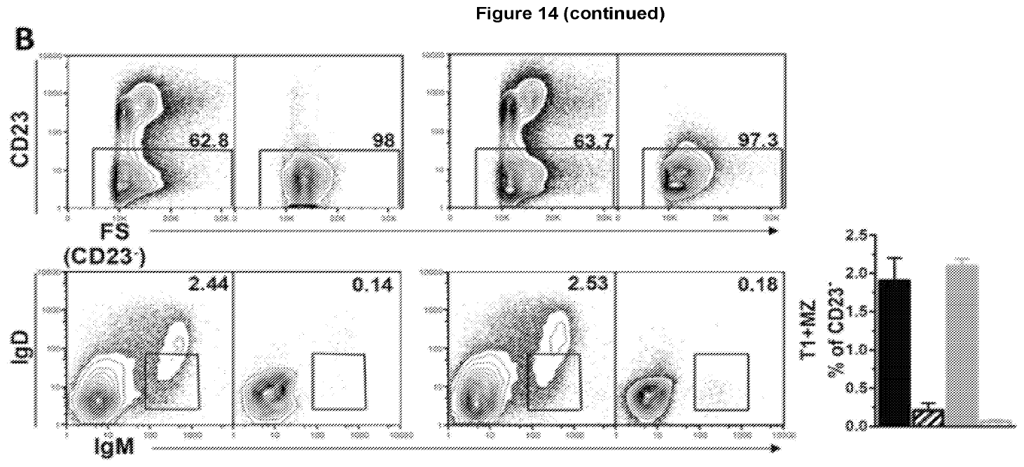

Lymphoid follicles and GC area and numbers were also measured by immunofluorescent staining 60 days after HCT. B-BCL6^{+/+}-no-GVHD recipients had intact lymphoid follicles and GCs, while B-BCL6^{-/-}-no-GVHD recipients had lymphoid follicles without GCs, as expected (FIG. 11E, top rows). Lymphoid follicles were damaged in both B-BCL6^{+/+}-cGVHD and B-BCL6^{-/-}-cGVHD recipients, and no GCs were detected in either recipient group (FIG. 11E, lower rows). Consistent with the well-preserved lymphoid follicles in both B-BCL6^{+/+} and B-BCL6^{-/-}-no-cGVHD recipients, comparable and abundant percentages of T2 B cells (CD23⁺IgD^{hi}IgM^{hi}CD21⁺), follicular B cells (CD23⁺IgD^{hi}IgM^{lo}CD21⁺), and T1/marginal zone B cells (CD23⁻IgD^{lo}IgM^{hi}) were detected by flow cytometry (FIGS. 14A, 14B). In a striking contrast, B-cell subsets were barely detectable in either B-BCL6^{+/+}-cGVHD or B-BCL6^{-/-}-cGVHD recipients (FIGS. 14A, 14B).

To further validate the absence of GCs observed with immunofluorescent staining, the percentage of Tfh and GC B cells in the spleen of recipients was measured by flow cytometry. Tfh cells were identified as CD4⁺CD19⁻PD-1^{hi}CXCR5^{hi}, and GC B cells were identified as CD19⁺Fas⁺GL7⁺, as previously described^{28,37}. Consistently, BCL6 deficiency in B cells led to ˜10-fold reduction in the percentage of Tfh and GC B cells in B-BCL6^{-/-}-no-GVHD recipients as compared to B-BCL6^{+/+}-no-GVHD recipients (FIGS. 11F, 11G). GVHD caused ˜65% reduction in the percentage of Tfh and GC B cells in B-BCL6^{+/+}-cGVHD recipients as compared to B-BCL6^{+/+}-no-GVHD recipients, although the percentages of these cells were still significantly higher than in B-BCL6^{-/-}-cGVHD or B-BCL6^{-/-}-no-GVHD recipients (FIGS. 11F, 11G). Taken together, these results indicate that GC formation is not required for induction of cGVHD and its associated lymphopenia.

### Example 3: Chronic GVHD is Linked with PSGL-1^{lo}CD4⁺ T-Cell Expansion

How extrafollicular CD4⁺ T- and B-cell interaction could mediate cGVHD was explored. PSGL-1 has been recently described as a new immune check point for T cells⁴¹, and extrafollicular CD4⁺ T cells in autoimmune mice have decreased expression of PSGL-1⁴². Extrafollicular CD4⁺ T cells were identified as CD44^{hi}CD62^{lo}PSGL-1^{lo}CD4⁺ (PSGL-1^{lo}CD4⁺) T cells with high level expression of ICOS, increased expression of CXCR4, and decreased expression of CXCR5, while TFH had increased expression of CXCR5. PSGL-1^{lo}CD4⁺ T cells also had increased production of IFN-γ, IL-13, and IL-21²⁵. Because expansion of PSGL-1^{lo}CD4⁺ T cells was observed in mice with autoimmune lupus²⁵, this population was tested to see whether it was also expanded during the pathogenesis of cGVHD. In the model used here, evidence of cutaneous GVHD began at ˜20 days after HCT and developed rapidly between days 30-45 after HCT (FIGS. 3A and 11A). Therefore, the percentage of PSGL-1^{lo}CD4⁺ T cells among total donor CD4⁺ T cells in the spleen, lung, and liver on days 21,30, and 45 after HCT was measured.

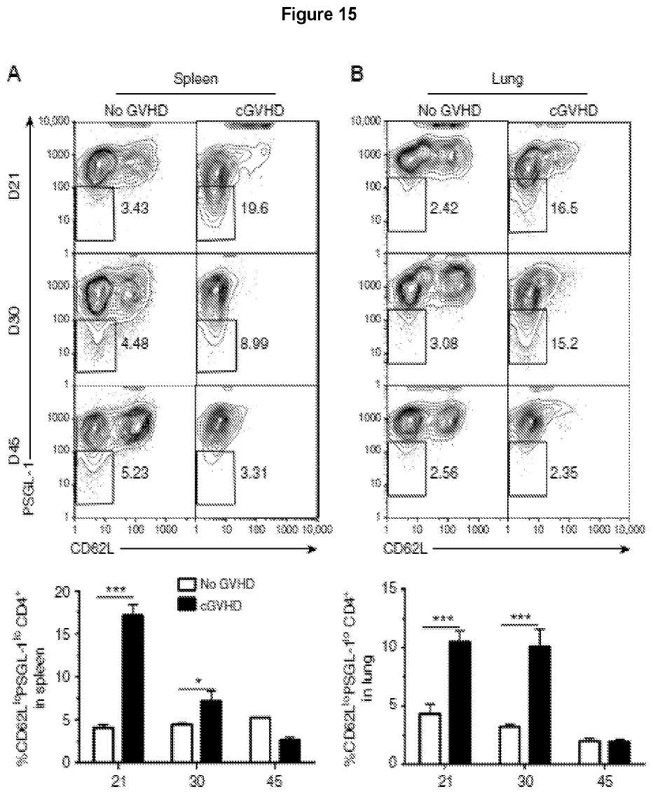

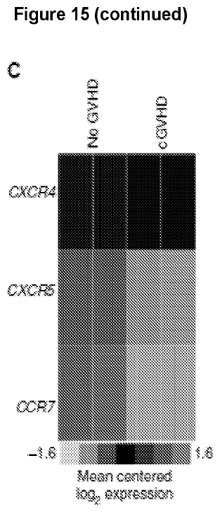

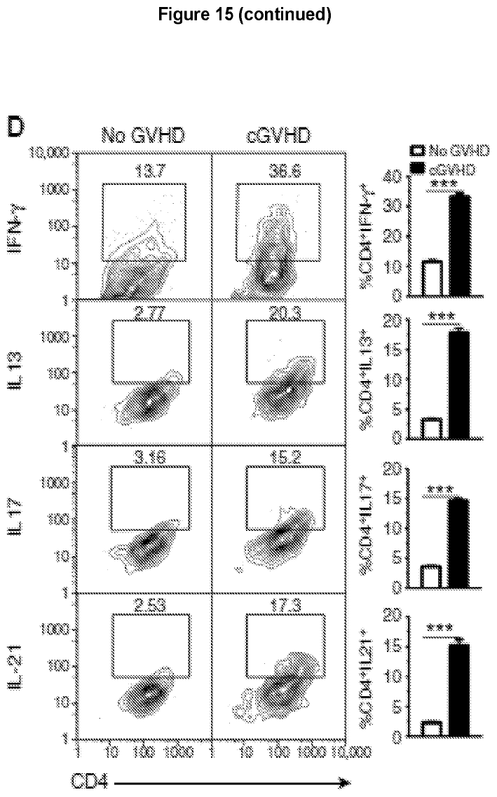

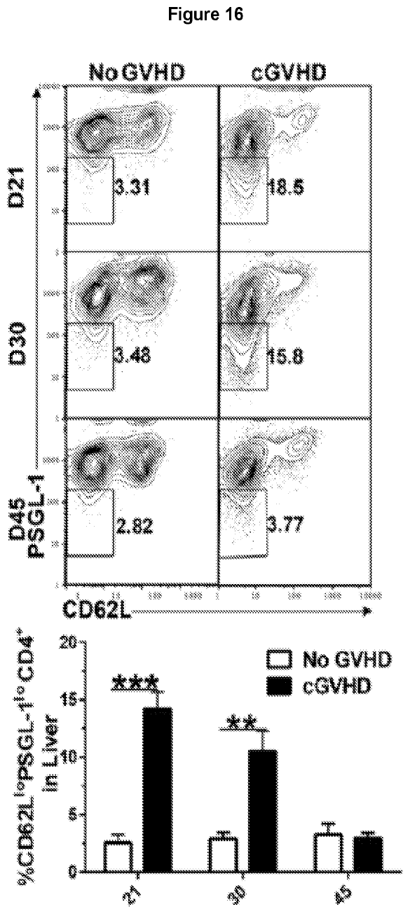

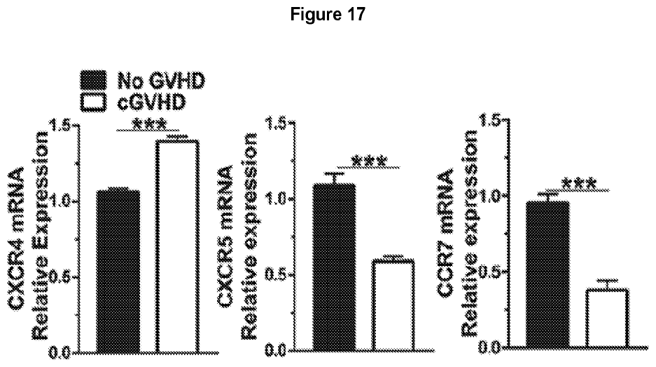

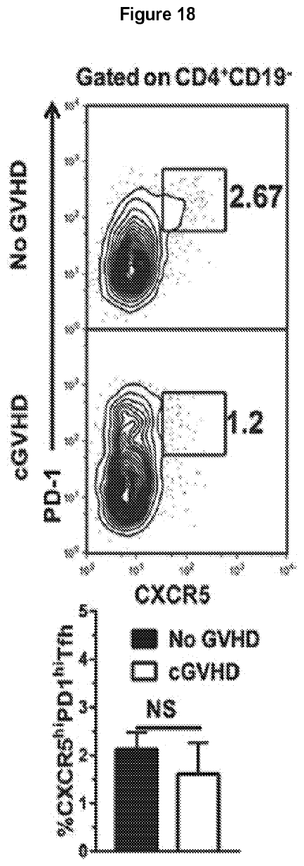

WT C57BL/6 donor grafts were transplanted into lethally irradiated BALB/c recipients as illustrated in FIG. 3. As compared with no-cGVHD recipients given TCD-BM cells, cGVHD recipients given spleen cells had ˜5-fold higher percentages of PSGL-1^{lo}CD4⁺ T cells in the spleen, lung, and liver on day 21 (FIGS. 15A, 15B and 16). The difference subsequently diminished in the spleen but was maintained in the lung and liver on day 30 and then disappeared by day 45 after HCT (FIGS. 15A, 15B and 16). The expanded PSGL-1^{lo}CD4⁺ T cells from the spleen of cGVHD recipients had slightly increased expression of CXCR4 but obviously decreased expression of CXCR5 and CCR7, as measured by RNA-seq analysis and suitably confirmed by real-time PCR (FIGS. 15C and 17). They also had 3-5-fold higher percentages of IFN-γ⁺, IL-13⁺, IL-17⁺, and IL-21⁺ cells (FIG. 15D). On the other hand, even at the peak expansion time of PSGL-1^{lo}CD4⁺ cells, day 21 after HCT, there was no obvious expansion of Tfh cells in the spleen of cGVHD recipients (FIG. 18). These results indicate that cGVHD development is associated with expansion of PSGL-1^{lo}CD4⁺ T cells but not Tfh cells.

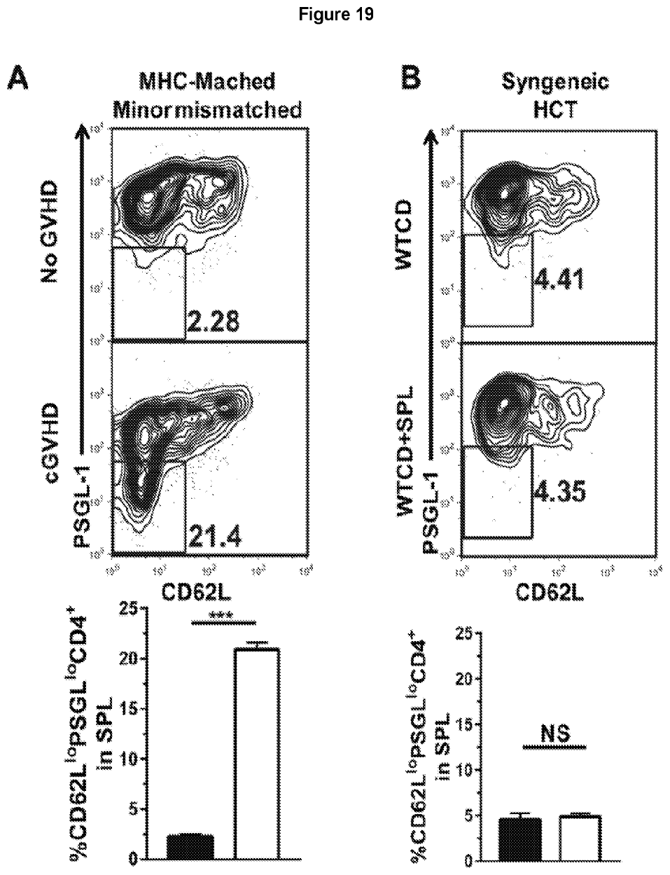

In addition, expansion of PSGL-1^{lo}CD4⁺ T cells was also observed in MHC-matched LP/J (H-2^{bc}) donor→C57BL/6 (H-2^{b}) model. Compared with no-cGVHD recipients given TCD-BM, cGVHD recipients given additional donor spleen cells had ˜10-fold increase in percentages of PSGL-1^{lo}CD4⁺ T cells in the spleen of recipients at 21 days after transplantation (FIG. 19). In contrast, there was no expansion of PSGL-1^{lo}CD4⁺ T cells in C57BL/6 recipients given syngeneic transplants (FIG. 19). These results indicate that expansion of PSGL-1^{lo}CD4⁺ T cells in cGVHD recipients is driven by MHC-matched and mismatched alloimmune responses but not by homeostatic expansion.

### Example 4: Blocking ICOS/ICOSL Binding Reduces PSGL-1^{lo}CD4⁺ T Expansion

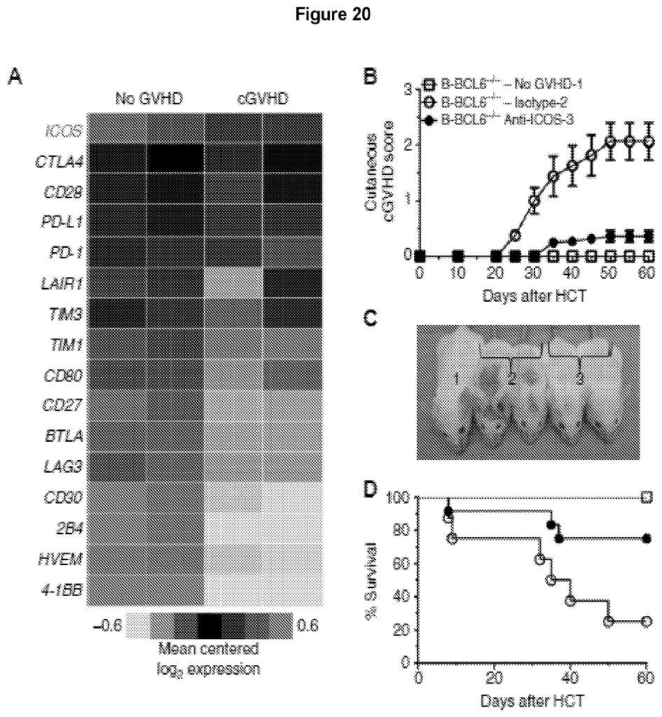

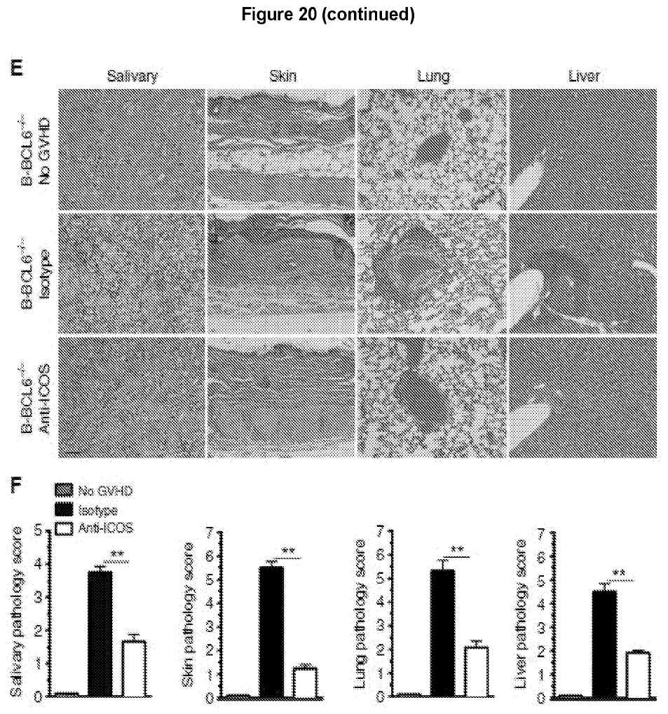

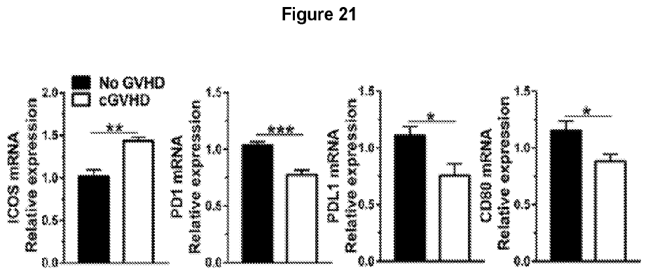

ICOS-dependent expansion of extrafollicular PSGL-1^{lo}CD4⁺ T cells is important in autoimmune pathogenesis in MRL^{lpr} mice that have defective GC formation²⁵. By RNA-seq analysis, it was found that PSGL-1^{lo}CD4⁺ T cells in cGVHD recipients with destruction of GCs upregulated expression of ICOS, although they downregulated expression of other costimulatory and coinhibitory molecules such as PD-1, CD80, and PD-L1 (FIG. 20A). The upregulation of ICOS and downregulation of PD-1, CD80, and PD-L1 were suitably confirmed by real-time PCR (FIG. 21). Whether blockade of ICOS and ICOS-ligand interaction prevented expansion of PSGL-1^{lo}CD4⁺ T and induction of cGVHD in recipients given B-BCL6^{-/-} grafts was tested, because these recipients have only extrafollicular CD4⁺ T and B interactions but no follicular GC formation. Accordingly, lethally irradiated BALB/c recipients were transplanted with spleen and TCD-BM from B-BCL6^{-/-} C57BL/6 donors and treated with blocking anti-ICOS mAb or control rat IgG2b (200 μg/mouse), every other day from days 0 to 45 after HCT. Recipients given TCD-BM alone were used as no-cGVHD controls. ICOS blockade markedly ameliorated cGVHD severity as judged by clinical score, survival, and histopathology (FIGS. 20B-20F).

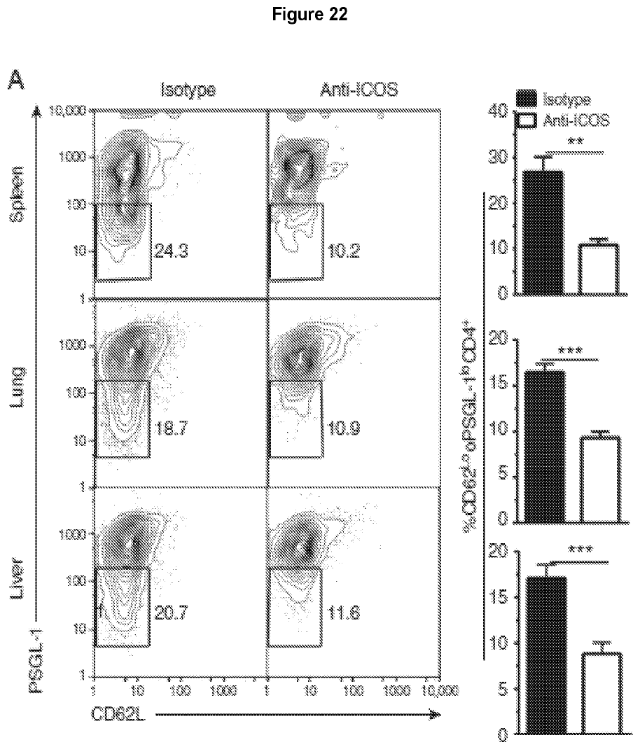

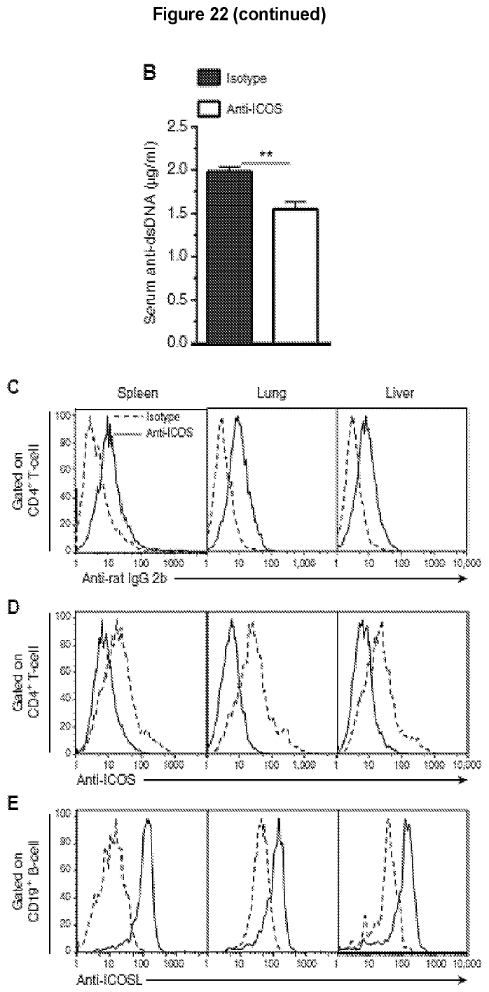

As compared to cGVHD recipients treated with control rat IgG2b, recipients with cGVHD prevented by ICOS blockade had a marked reduction in the percentage of PSGL-1^{lo}CD4⁺ T cells in the spleen, lung, and liver (FIG. 22A), which was associated with reduction of serum anti-dsDNA-IgG concentrations (FIG. 22B). In addition, whether the reduced percentages of PSGL-1^{lo}CD4⁺ T cells were associated with specific blockade of ICOS and ICOS-ligand interaction was tested. As compared with CD4⁺ T cells from recipients treated with control rat IgG2b, the CD4⁺ T cells from recipients treated with anti-ICOS mAb had reduced direct anti-ICOS staining with increased anti-rat IgG2b staining (FIGS. 22C, 22D), indicating that anti-ICOS (IgG2b) mAb bound to the ICOS expressing CD4⁺ T cells and blocked anti-ICOS staining. Furthermore, anti-ICOS mAb treatment led to upregulated expression of ICOS-L by B cells, indicating disruption of ICOS-ICOSL interaction between CD4⁺ T and B cells (FIG. 22E). Taken together, these results indicate that in recipients without GC formation, blockade of ICOS/ICOSL interaction reduces extrafollicular PSGL-1^{lo}CD4⁺ T-cell expansion, reduces autoantibody production, and ameliorates cGVHD. These results suggest that extrafollicular PSGL-1^{lo}CD4⁺ T cells have a critical function in the pathogenesis of cGVHD.

### Example 5: Donor BCL6 Deficiency Reduces PSGL-1^{lo}CD4⁺ T Expansion

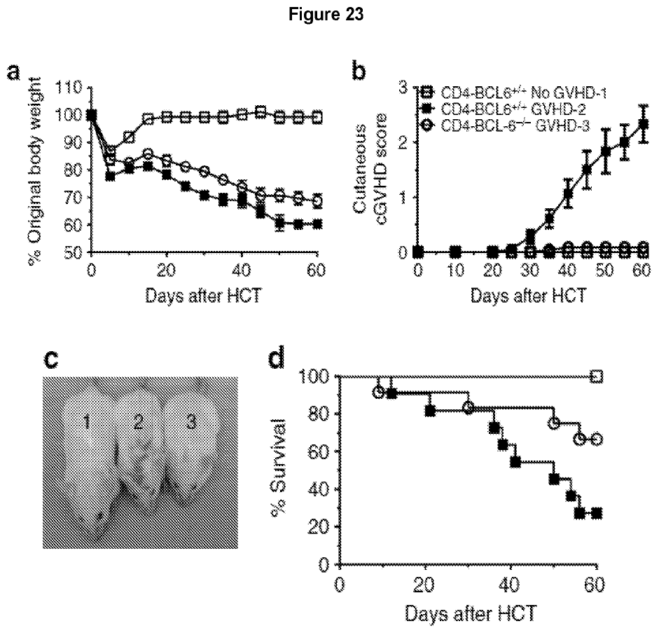

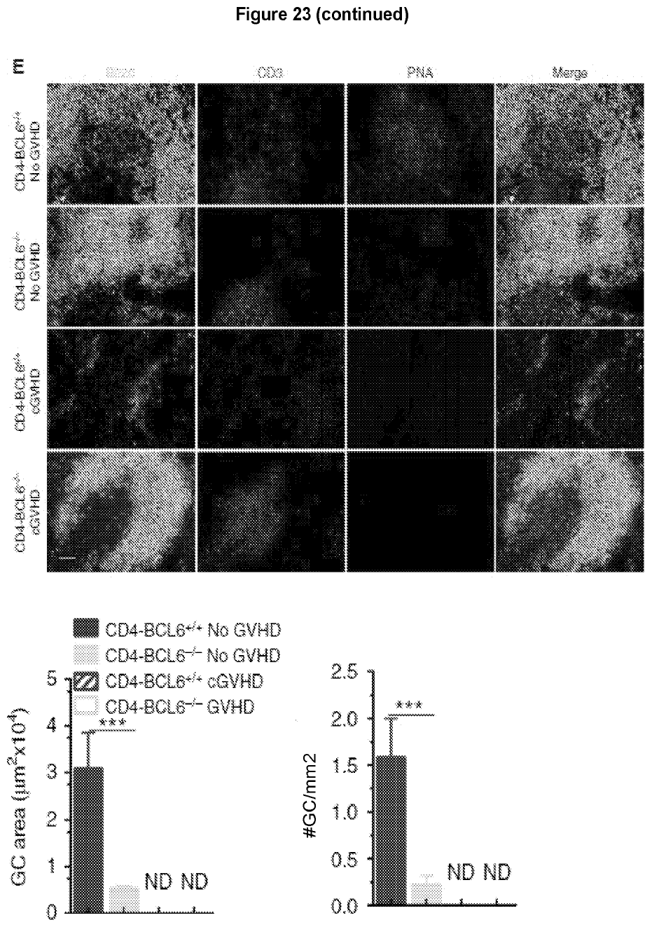

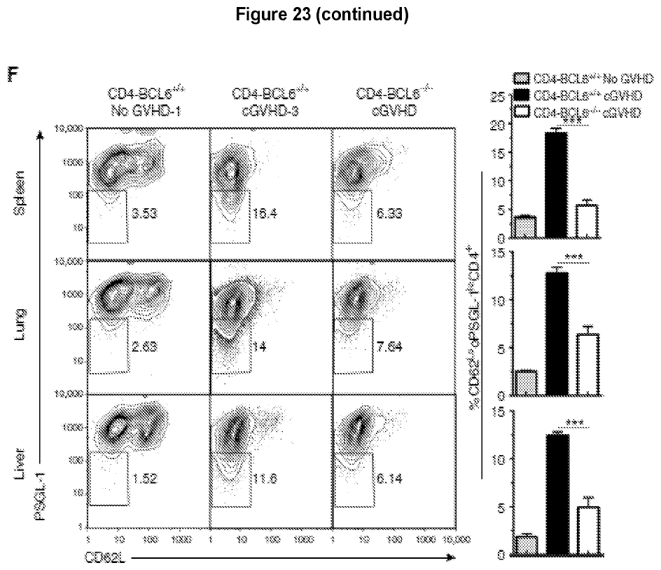

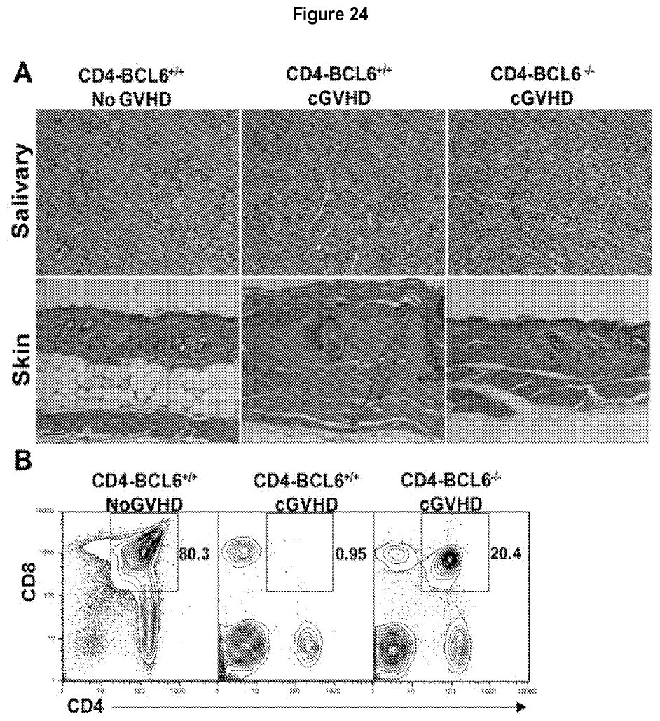

CD4⁺ T-cell expression of BCL6 is necessary for extrafollicular CD4⁺ T- and B-cell interactions in non-autoimmune mice¹⁹. Therefore, the impact of BCL6 deficiency in donor CD4⁺ T cells on induction of autoimmune-like cGVHD and expansion of PSGL-1^{lo}CD4⁺ T cells was tested. Accordingly, TCD-BM (2.5×10⁶) and spleen cells (1×10⁶) from BCL6^{fl/fl} CD4-Cre-C57BL/6 (CD4-BCL6^{-/-}) donors that have BCL6 deficiency specifically in CD4⁺ T cells or from control BCL6^{fl/fl} C57BL/6 (CD4-BCL6^{+/+}) littermates were transplanted into lethally irradiated BALB/c recipients. Recipients given TCD-BM alone from either donor showed no signs of GVHD (no-GVHD) and were combined into a single control group. Recipients given TCD-BM and spleen cells from control CD4-BCL6^{+/+} donors developed severe cGVHD with weight loss and hair loss, and ˜75% died within 60 days after HCT (FIGS. 23A-23D). In contrast, recipients given TCD-BM and spleen cells from CD4-BCL6^{-/-} donors had weight loss and ˜30% mortality but no signs of cutaneous cGVHD (FIGS. 23A-23D). In addition, CD4-BCL6^{-/-}-cGVHD recipients showed little skin tissue damage by histopathology and had reduced damage in the thymus as indicated by higher numbers of CD4⁺CD8⁺ thymocytes, but the extent of salivary gland damage was similar as compared to CD4-BCL6^{+/+}-cGVHD recipients (FIG. 24). These results indicate that BCL6 deficiency in donor CD4⁺ T cells prevents cGVHD-associated damage in the skin and decreases damage in the thymus.

In the spleen of recipients given CD4-BCL6^{-/-} TCD-BM, GCs were not completely absent, but their size and numbers were decreased by more than 85% as compared to recipients given CD4-BCL6^{+/+} TCD-BM (FIG. 23E). CD4-BCL6^{+/+}-cGVHD recipients had no lymphoid follicles or GCs, while CD4-BCL6^{-/-}-cGVHD recipients had lymphoid follicles but no GCs (FIG. 23E). Compared with CD4-BCL6^{+/+}-cGVHD recipients, CD4-BCL6^{-/-}-cGVHD recipients had lower numbers of PSGL-1^{lo}CD4⁺ T cells in the spleen, lung, and liver (FIG. 23F).

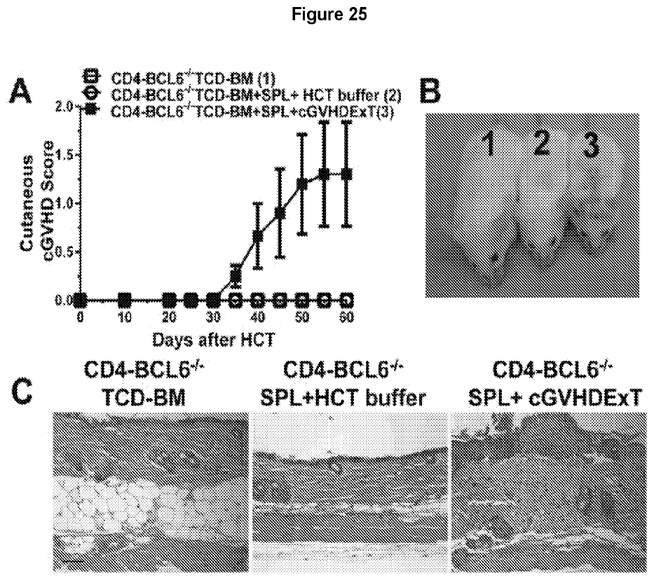

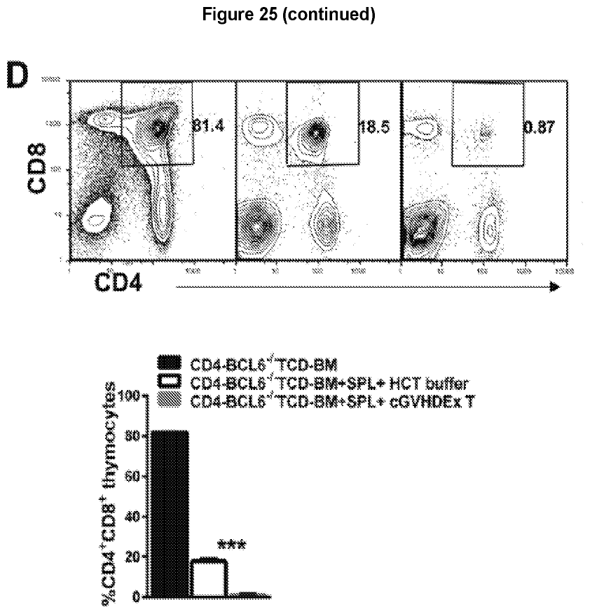

To further test the role of PSGL1^{lo}CD4⁺ T cells in skin and thymus damage in cGVHD recipients, sorted PSGL1loCD4+ T cells (1×10⁶) from the spleen of cGVHD recipients given B-BCL6^{-/-} transplants at 21 days after HCT were injected into recipients given CD4-BCL6^{-/-} transplants. The control recipients were given transplantation buffer only. Injection of the PSGL1^{lo}CD4⁺ T cells augmented clinical signs of cutaneous GVHD and skin pathology and exacerbated loss of CD4⁺CD8⁺ thymocytes (FIG. 25). These results indicate that expansion of extrafollicular PSGL-1^{lo}CD4⁺ T cells in cGVHD recipients is BCL6-dependent and that prevention of extrafollicular CD4⁺ T-cell expansion can effectively prevent induction of cutaneous cGVHD, although other manifestations of cGVHD persist.

### Example 6: Donor Stat3 Deficiency Reduces PSGL-1^{lo}CD4⁺ T Expansion

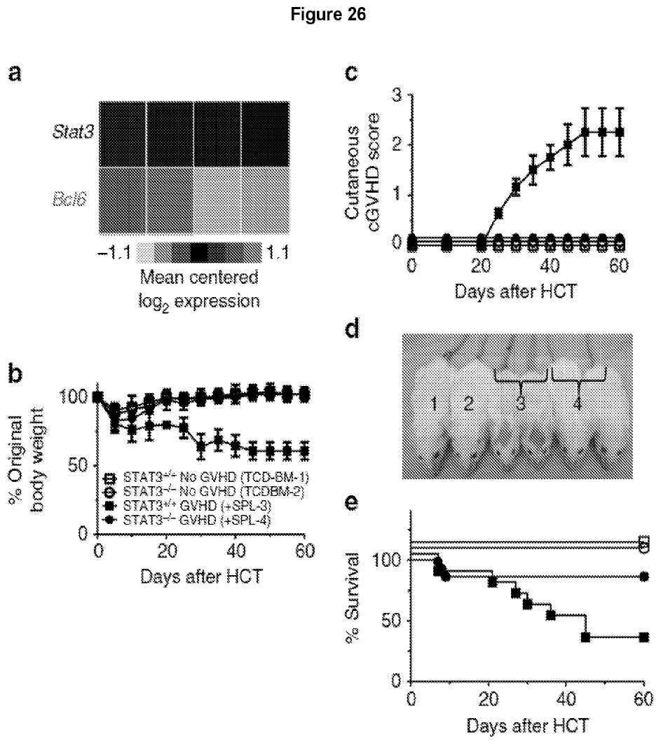

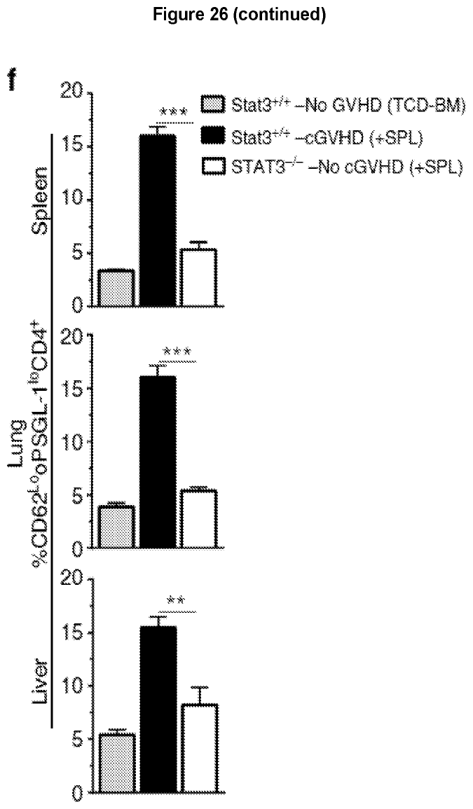

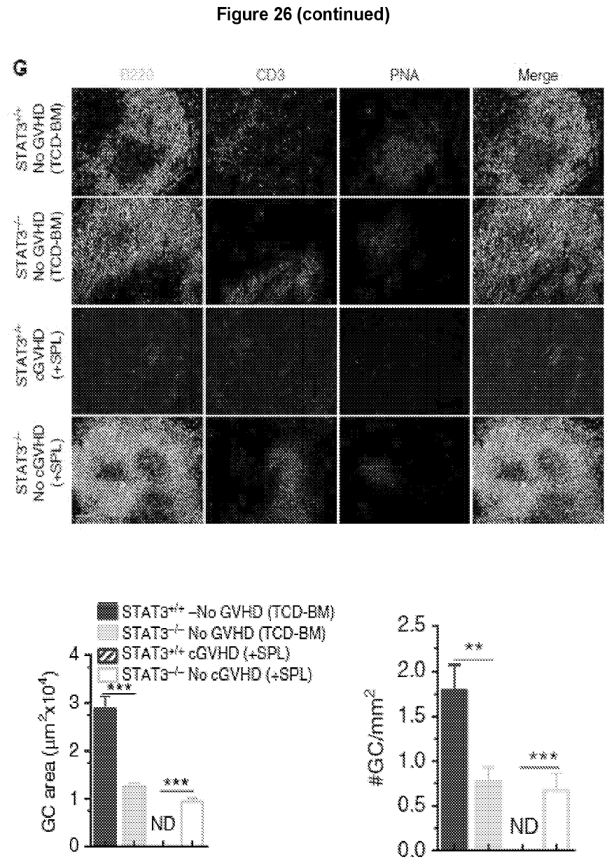

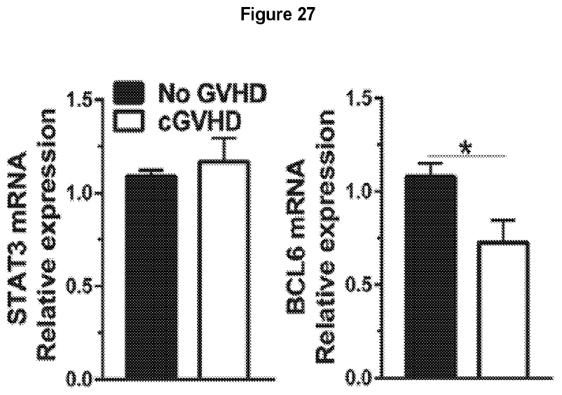

Transcription factors, Stat3 and BCL6, are both expressed by prefollicular CD4⁺ T cells and are required for differentiation of Tfh and GC formation^{14,39}. Therefore, RNA-seq analysis was used to compare the expression levels of these transcription factors in PSGL-1^{lo}CD4⁺ T cells from the spleen of no-GVHD recipients given WT-TCD-BM and cGVHD recipients given WT-TCD-BM and spleen cells. Although BCL6 was required for expansion of PSGL-1^{lo}CD4⁺ T cells in cGVHD recipients (FIG. 23), expression of BCL6 appeared to be downregulated in those cells (FIG. 26A). However, expression of Stat3 in those cells was slightly increased although not significant, as measured by RNA-seq and suitably confirmed by real-time PCR analysis (FIGS. 26A and 27). These data suggested a possible function of Stat3 in maintaining the expansion of PSGL-1^{lo}CD4⁺ T cells and the induction of cGVHD. Therefore, PSGL-1^{lo}CD4⁺ T-cell expansion and cGVHD induction were evaluated by Stat3-deficient donor CD4⁺ T cells.

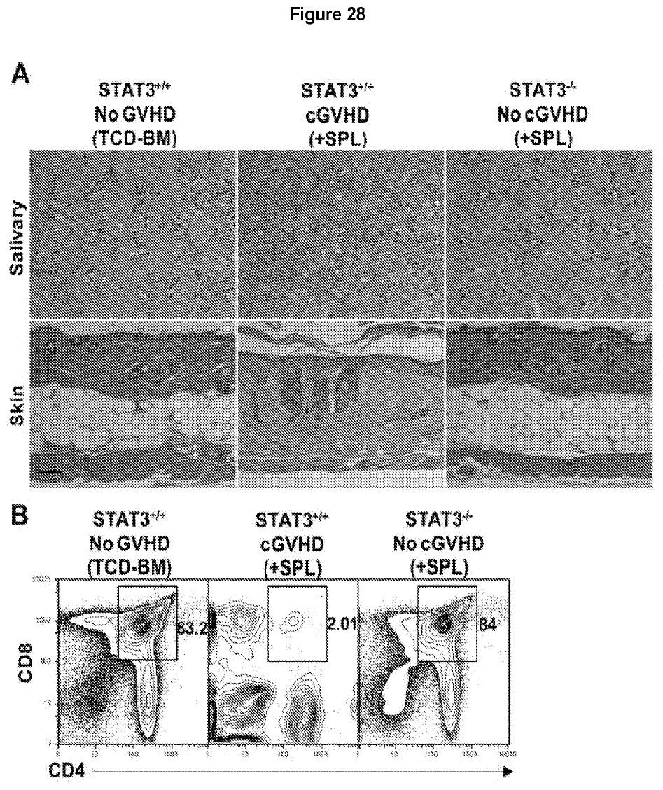

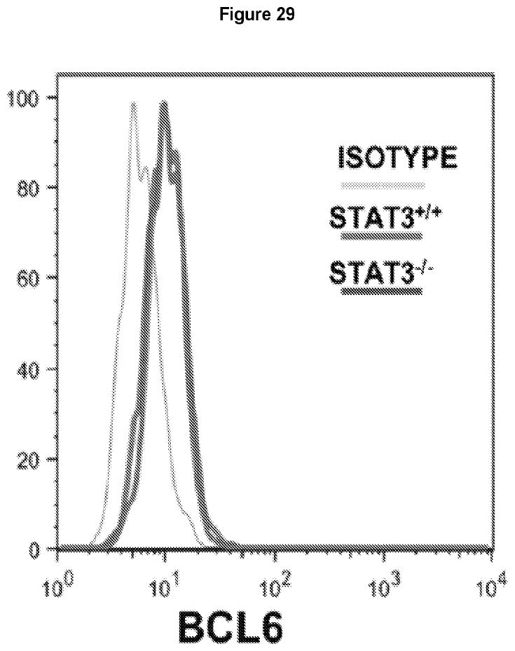

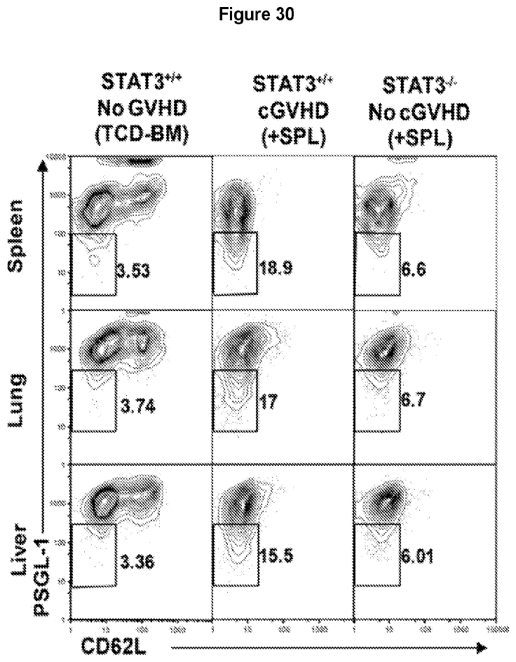

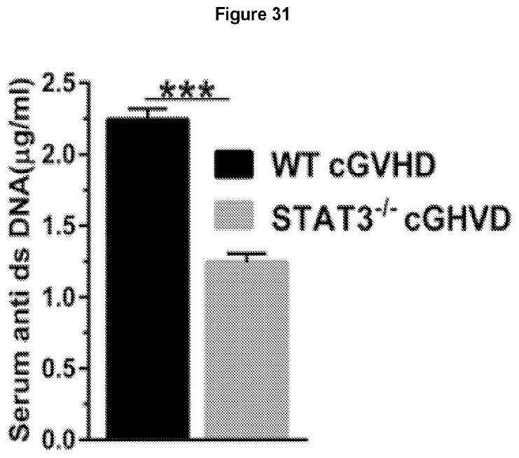

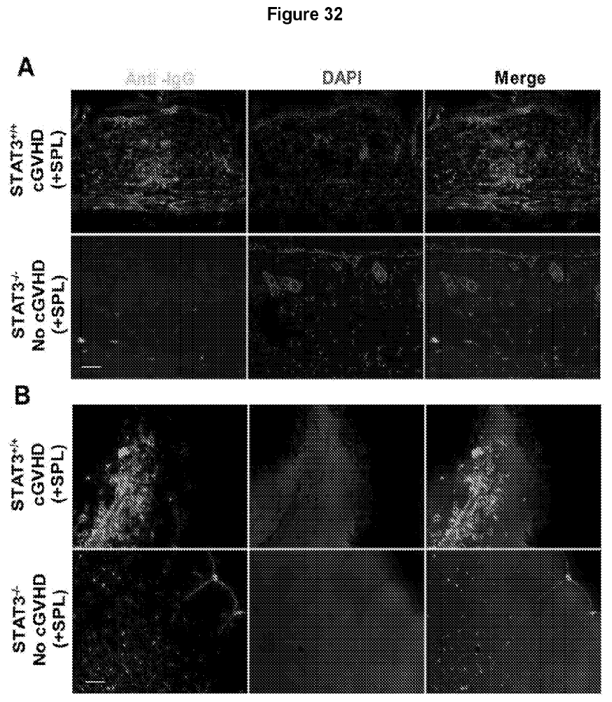

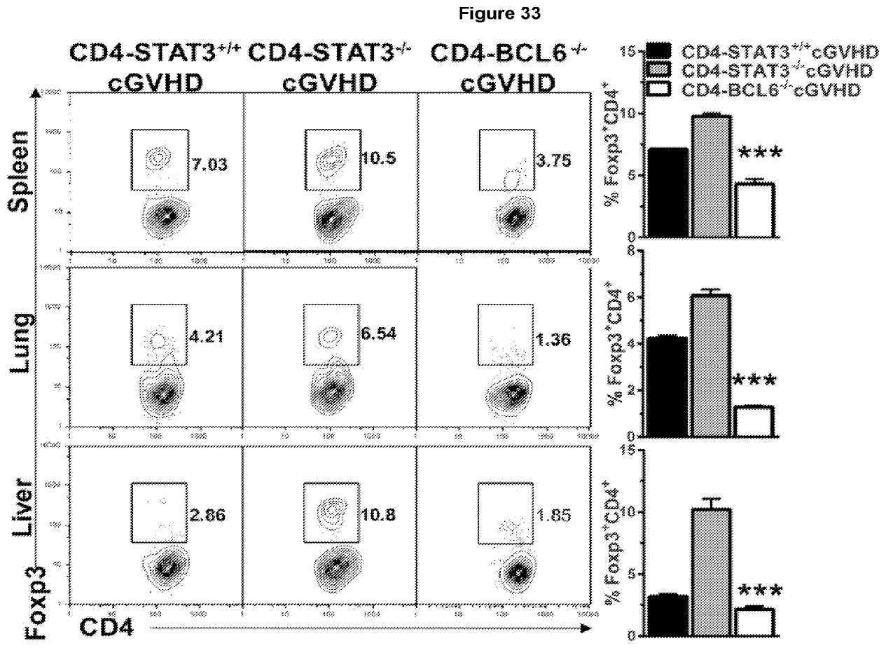

Accordingly, TCD-BM (2.5×10⁶) and spleen cells (1×10⁶) from Stat3^{fl/fl} CD4-Cre-057BL/6 donors that have Stat3 deficiency specifically in CD4⁺ T cells (CD4-Stat3^{-/-}) or from control Stat3^{fl/fl} C57BL/6 (CD4-Stat3^{+/+}) littermates were transplanted into lethally irradiated BALB/c recipients. Recipients given CD4-Stat3^{-/-} transplants had no clinical evidence of cGVHD in the skin and no mortality beyond day 15 after HCT (FIGS. 26B-26E). Prevention of cGVHD in recipients given CD4-Stat3^{-/-} grafts was confirmed by lack of damage in the thymus, as judged by normal percentages of CD4⁺CD8⁺ thymocytes and the lack of damage in the skin and salivary glands (FIG. 28). Although Stat3^{-/-} PSGL-1^{lo}CD4⁺ T cells expressed similar levels of BCL6 as compared to wildtype controls (FIG. 29), prevention of cGVHD in recipients given Stat3^{-/-} transplants was associated with lower percentages of PSGL-1^{lo}CD4⁺ T cells in the spleen, lung, and liver tissues (FIGS. 26F and 30). The prevention of cGVHD was also associated with lower serum concentrations of anti-dsDNA-IgG (FIG. 31) and less IgG deposition in the skin and thymus (FIGS. 32A, 32B). In addition, recipients given CD4-Stat3^{-/-} grafts had increased percentages of Foxp3+CD4⁺ Treg cells as compared to cGVHD recipients given WT grafts or CD4-BCL6^{-/-} grafts (P<0.001, FIG. 33).

GC formation in recipients given CD4-Stat3^{-/-} TCD-BM was not completely absent, but the size and numbers of GCs were reduced by more than 50% (FIG. 26G) as compared to no-GVHD recipients given CD4-Stat3^{+/+} TCD-BM. Chronic GVHD recipients given CD4-Stat3^{+/+} TCD-BM and spleen cells completely lost lymphoid follicles and GCs (FIG. 26G). In contrast, cGVHDfree recipients given CD4-Stat3^{-/-} TCD-BM and spleen cells had preserved lymphoid follicles, and their GC size and numbers were similar to those in no-GVHD recipients given CD4-Stat3^{-/-} TCD-BM alone (FIG. 26G).

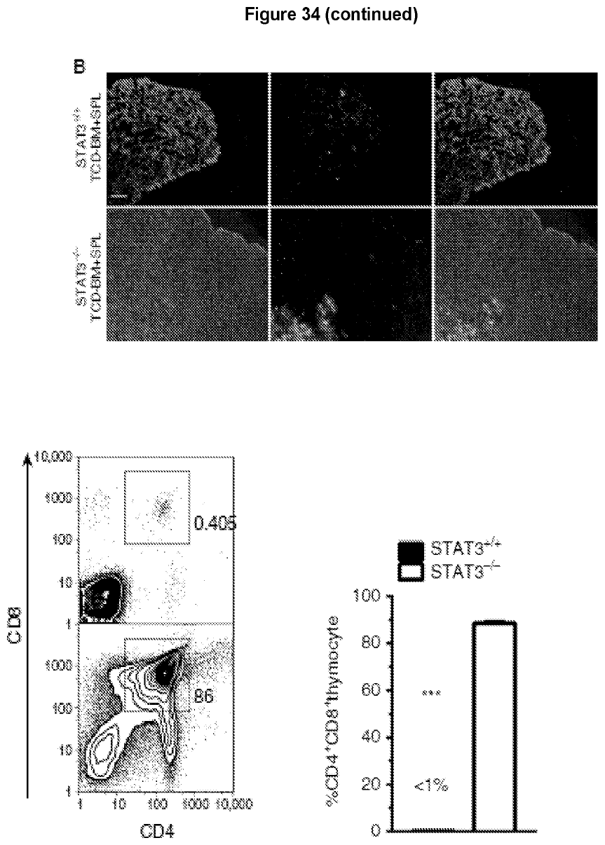

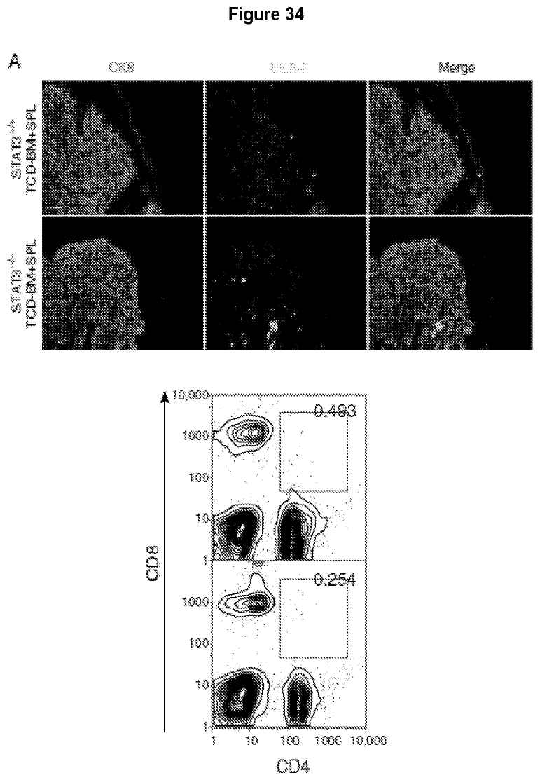

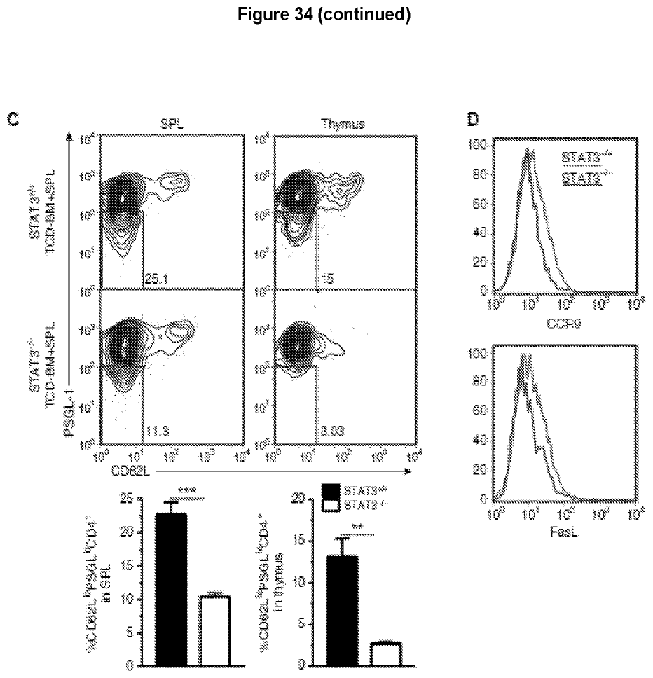

The function of PSGL-1^{lo}CD4⁺ T cells in thymus damage early after HCT in recipients given Stat3^{+/+} or Stat3^{-/-} transplants was evaluated because CD4⁺ T cells are important in damaging thymus via expression of FasL early after allogenic HCT⁴³. Moreover, thymus damage is important in persistence of chronic GVHD^{7,9}. First, thymus damage was measured with immunofluorescent staining of thymic cortical (CK8) and medullary (UEA-1) epithelial cells and percentage of CD4⁺CD8⁺ thymocytes was determined at 10 and 30 days after HCT. Thymus damage was similar in recipients given CD4-Stat3^{+/+} or CD4-Stat3^{-/-} transplants at 10 days after HCT, with loss of UEA-1⁺ medullary epithelial cells and CD4⁺CD8⁺ thymocytes (FIG. 34A). By 30 days after HCT, the difference become obvious, and the recovery of cortical and medullary epithelial cells and CD4⁺CD8⁺ thymocytes in recipients given Stat3^{-/-} transplants was much better than in recipients given Stat3^{+/+} transplants (FIG. 34B). The better thymic recovery in recipients given Stat3^{-/-} transplants was associated with much lower numbers of PSGL-1^{lo}CD4⁺ T cells in the spleen and marked reduction of infiltration in the thymus (FIG. 34C). The PSGL-1^{lo}CD4⁺ T cells in the spleen of recipients also expressed lower levels of CCR9 and FasL (FIG. 34D). CCR9 enables CD4⁺ T migration into thymus⁴⁴ and FasL expressed by the CD4⁺ T cells can mediate thymus damage⁴³.

Taken together, these results indicate that deficiency of Stat3 in donor CD4⁺ T cells results in (1) marked reduction of expansion of extrafollicular PSGL-1^{lo}CD4⁺ T cells and reduction of their infiltration of thymus; (2) lower production of serum IgG autoantibodies and decreased IgG deposition in GVHD target tissues such as skin and thymus. Finally, reduction of PSGL-1^{lo}CD4⁺ T infiltration and decreased IgG deposition allow recovery of the thymus early after HCT with production of Foxp3⁺ Treg cells, leading to full prevention of cGVHD.

### Example 7: Stat3 Knockdown in Mice

The sequences of the shRNAs used in this experiment are:

shSTAT3-1 (target sequence #1):
(SEQ ID NO: 19)
GTGTCAGATCACATGGGCTA
(antisense UAGCCCAUGUGAUCUGACAC, SEQ ID NO: 20);
shSTAT3-2 (target sequence #2):
(SEQ ID NO: 21)
GCAGCTGAACAACATGTCAT
(antisense AUGACAUGUUGUUCAGCUGC, SEQ ID NO: 22);
and
shRNA-ctrl (shRNA control sequence):
(antisense, SEQ ID NO: 23)
AGCGUUCUACACUCGACGUACU.

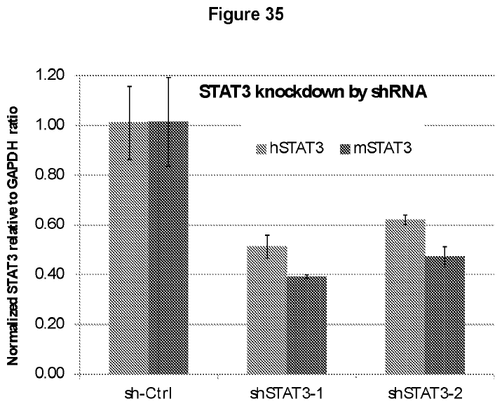

Human U6 promoter driven shRNA were cloned into LV-EF1a-EGFP to create lentiviral vectors: LV-U6-shRNA (STAT3 or Control)-EF1a-EGFP and tested for STAT3 knockdown. Lentiviral vectors were transfected into HEK293 cells to measure human STAT3 (hSTAT3) knockdown, and mouse embryonic fibroblast (MEF) cells to measure mouse STAT3 (mSTAT3) knockdown, as shown in FIG. 35. 48 hours after transfection, total RNA was isolated and used for qPCR measurement.

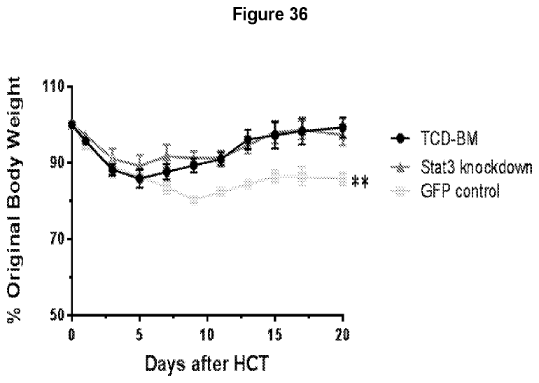

Subsequently lentiviruses were produced using the lentiviral vectors described above. These lentiviruses were used to transduce stimulated T cells, and EGFP positive T cells were sorted for mouse experiments, as shown in FIG. 36. Lethally irradiated BALB/c recipients were transplanted with sorted EGFP⁺ cells (1-2×10⁶) and TCD-BM (5×10⁶) from C57BL/6 donor. FIG. 36 shows the body weight change over a period of up to 20 days after HCT. The TCB-BM and Stat3 knockdown mice demonstrated good recovery of body weight loss in about a week after HCT compared to the control.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. M. Horowitz, M. Uses and growth of hematopoietic cell transplantation. In Thomas' Hematopoietic Cell Transplantation 5th edn, (eds Stephen J., Forman R. S. N., Antin, J. H., Appelbaum, F. R.) (2016).
2. Cooke K. R., et al. The biology of chronic graft-versus-host disease: a task force report from the national institutes of health consensus development project on criteria for clinical trials in chronic graft-versus-host disease. Biol. Blood Marrow Transplant 23, 211-234 (2016).
3. Flowers, M. E. & Martin, P. J. How we treat chronic graft-versus-host disease. Blood 125, 606-615 (2015).
4. Filipovich, A. H. et al. National Institutes of Health consensus development project on criteria for clinical trials in chronic graft-versus-host disease: I. Diagnosis and staging working group report. Biol. Blood Marrow Transplant 11, 945-956 (2005).
5. Zhao, D. et al. Alloimmune response results in expansion of autoreactive donor CD4+ T cells in transplants that can mediate chronic graft-versus-host disease. J. Immunol. 186, 856-868 (2011).
6. Young, J. S. et al. Donor B cells in transplants augment clonal expansion and survival of pathogenic CD4+ T cells that mediate autoimmune-like chronic graft-versus-host disease. J. Immunol. 189, 222-233 (2012).
7. Wu, T. et al. Thymic damage, impaired negative selection, and development of chronic graft-versus-host disease caused by donor CD4+ and CD8+ T cells. J. Immunol. 191, 488-499 (2013)
8. Zhang, C. et al. Donor CD4+ T and B cells in transplants induce chronic graftversus-host disease with autoimmune manifestations. Blood 107, 2993-3001 (2006).
9. Jin, H. et al. Antibodies from donor B cells perpetuate cutaneous chronic graftversus-host disease in mice. Blood 127, 2249-2260 (2016).
10. Rozans, M. K., Smith, B. R., Burakoff, S. J. & Miller, R. A. Long-lasting deficit of functional T cell precursors in human bone marrow transplant recipients revealed by limiting dilution methods. J. Immunol. 136, 4040-4048 (1986).
11. Dulude, G., Roy, D. C. & Perreault, C. The effect of graft-versus-host disease on T cell production and homeostasis. J. Exp. Med. 189, 1329-1342 (1999).
12. Shlomchik, M. J., Craft, J. E. & Mamula, M. J. From T to B and back again: positive feedback in systemic autoimmune disease. Nat. Rev. Immunol. 1, 147-153 (2001).
13. Steele, D. J. et al. Two levels of help for B cell alloantibody production. J. Exp. Med. 183, 699-703 (1996).
14. Crotty, S. T follicular helper cell differentiation, function, and roles in disease. Immunity 41, 529-542 (2014).
15. Basso, K. & Dalla-Favera, R. Roles of BCL6 in normal and transformed germinal center B cells. Immunol. Rev. 247, 172-183 (2012).
16. Veerman, K. M. et al. Interaction of the selectin ligand PSGL-1 with chemokines CCL21 and CCL19 facilitates efficient homing of T cells to secondary lymphoid organs. Nat. Immunol. 8, 532-539 (2007).
17. Kaji, T. et al. Distinct cellular pathways select germline-encoded and somatically mutated antibodies into immunological memory. J. Exp. Med. 209, 2079-2097 (2012).
18. Toyama, H. et al. Memory B cells without somatic hypermutation are generated from Bcl6-deficient B cells. Immunity 17, 329-339 (2002).
19. Lee, S. K. et al. B cell priming for extrafollicular antibody responses requires Bcl-6 expression by T cells. J. Exp. Med. 208, 1377-1388 (2011).
20. Tangye, S. G., Ma, C. S., Brink, R. & Deenick, E. K. The good, the bad and the ugly-TFH cells in human health and disease. Nat. Rev. Immunol. 13, 412-426 (2013).
21. Haynes, N. M., Allen, C. D., Lesley, R., Ansel, K. M., Killeen, N. & Cyster, J. G. Role of CXCR5 and CCR7 in follicular Th cell positioning and appearance of a programmed cell death gene-1high germinal center-associated subpopulation. J. Immunol. 179, 5099-5108 (2007).
22. Ram iscal, R. R. & Vinuesa, C. G. T-cell subsets in the germinal center. Immunol. Rev. 252, 146-155 (2013).
23. Bentebibel, S. E. et al. Induction of ICOS+CXCR3+CXCR5+TH cells correlates with antibody responses to influenza vaccination. Sci. Transl. Med. 5, 176ra132 (2013).
24. Linterman, M. A. et al. Follicular helper T cells are required for systemic autoimmunity. J. Exp. Med. 206, 561-576 (2009).
25. Odegard, J. M. et al. ICOS-dependent extrafollicular helper T cells elicit IgG production via IL-21 in systemic autoimmunity. J. Exp. Med. 205, 2873-2886 (2008).
26. Szabo, K. et al. Follicular helper T cells may play an important role in the severity of primary Sjogren's syndrome. Clin. Immunol. 147, 95-104 (2013).
27. Liarski, V. M. et al. Cell distance mapping identifies functional T follicular helper cells in inflamed human renal tissue. Sci. Transl. Med. 6, 230ra246 (2014).
28. Flynn, R. et al. Increased T follicular helper cells and germinal center B cells are required for cGVHD and bronchiolitis obliterans. Blood 123, 3988-3998 (2014).
29. Dubovsky, J. A. et al. Ibrutinib treatment ameliorates murine chronic graftversus-host disease. J. Clin. Invest. 124, 4867-4876 (2014).
30. Srinivasan, M. et al. Donor B-cell alloantibody deposition and germinal center formation are required for the development of murine chronic GVHD and bronchiolitis obliterans. Blood 119, 1570-1580 (2012).
31. Knorr, D. A. et al. Loss of T follicular helper cells in the peripheral blood of patients with chronic graft-versus-host disease. Biol. Blood Marrow Transplant 22, 825-833 (2016).
32. Forcade, E. et al. Circulating T follicular helper cells with increased function during chronic graft-versus-host disease. Blood 127, 2489-2497 (2016).
33. Suzuki, I. et al. Immunoglobulin heavy chain variable region gene usage in bone marrow transplant recipients: lack of somatic mutation indicates a maturational arrest. Blood 87, 1873-1880 (1996).
34. Glas, A. M. et al. B-cell-autonomous somatic mutation deficit following bone marrow transplant. Blood 96, 1064-1069 (2000).
35. Li, X. et al. Loss of B7-H1 expression by recipient parenchymal cells leads to expansion of infiltrating donor CD8+ T cells and persistence of graft-versus host disease. J. Immunol. 188, 724-734 (2012).
36. Socie, G. & Ritz, J. Current issues in chronic graft-versus-host disease. Blood 124, 374-384 (2014).
37. Sage, P. T., Francisco, L. M., Carman, C. V. & Sharpe, A. H. The receptor PD-1 controls follicular regulatory T cells in the lymph nodes and blood. Nat. Immunol. 14, 152-161 (2013).
38. Zhang, Y., McCormick, L. L., Desai, S. R., Wu, C. & Gilliam, A. C. Murine sclerodermatous graft-versus-host disease, a model for human scleroderma: cutaneous cytokines, chemokines, and immune cell activation. J. Immunol. 168, 3088-3098 (2002).
39. Nurieva, R. I. et al. Bcl6 mediates the development of T follicular helper cells. Science 325, 1001-1005 (2009).
40. Geng, H. et al. Self-enforcing feedback activation between BCL6 and pre-B cell receptor signaling defines a distinct subtype of acute lymphoblastic leukemia. Cancer Cell 27, 409-425 (2015).
41. Tinoco, R. et al. PSGL-1 is an immune checkpoint regulator that promotes T cell exhaustion. Immunity 44, 1190-1203 (2016).
42. Weinstein, J. S., Hernandez, S. G. & Craft, J. T cells that promote B-Cell maturation in systemic autoimmunity. Immunol. Rev. 247, 160-171 (2012).
43. Na, I. K. et al. The cytolytic molecules Fas ligand and TRAIL are required for murine thymic graft-versus-host disease. J. Clin. Invest. 120, 343-356 (2010).
44. Zabel, B. A. et al. Human G protein-coupled receptor GPR-9-6/CC chemokine receptor 9 is selectively expressed on intestinal homing T lymphocytes, mucosal lymphocytes, and thymocytes and is required for thymus-expressed chemokine-mediated chemotaxis. J. Exp. Med. 190, 1241-1256 (1999).
45. Munoz-Fernandez, R. et al. Follicular dendritic cells are related to bone marrow stromal cell progenitors and to myofibroblasts. J. Immunol. 177, 280-289 (2006).
46. Li, J. et al. Roles of CD28, CTLA4, and inducible costimulator in acute graftversus-host disease in mice. Biol. Blood Marrow Transplant. 17, 962-969 (2011).
47. Fujimura, J. et al. Contribution of B7RP-1/ICOS co-stimulation to lethal acute GVHD. Pediatr. Transplant. 14, 540-548 (2010).
48. Mollweide, A. et al. Only therapeutic ICOS:ICOSL blockade alleviates acute graft versus host disease. Klin. Padiatr. 221, 344-350 (2009).
49. Vu Van, D. et al. Local T/B cooperation in inflamed tissues is supported by T follicular helper-like cells. Nat. Commun. 7, 10875 (2016).
50. Rao, D. A. et al. Pathologically expanded peripheral T helper cell subset drives B cells in rheumatoid arthritis. Nature 542, 110-114 (2017).
51. Drobyski, W. R. et al. Tocilizumab for the treatment of steroid refractory graftversus-host disease. Biol. Blood Marrow Transplant. 17, 1862-1868 (2011).
52. Chen, X. et al. Blockade of interleukin-6 signaling augments regulatory T-cell reconstitution and attenuates the severity of graft-versus-host disease. Blood 114, 891-900 (2009).
53. Lu, D. et al. The phosphatase DUSP2 controls the activity of the transcription activator STAT3 and regulates TH17 differentiation. Nat. Immunol. 16, 1263-1273 (2015).
54. Laurence, A. et al. STAT3 transcription factor promotes instability of nTreg cells and limits generation of iTreg cells during acute murine graft-versus-host disease. Immunity 37, 209-222 (2012).
55. Vinuesa, C. G., Linterman, M. A., Yu, D. & MacLennan, I. C. Follicular helper T cells. Annu. Rev. Immunol. 34, 335-368 (2016).
56. Radojcic, V. et al. STAT3 signaling in CD4+ T cells is critical for the pathogenesis of chronic sclerodermatous graft-versus-host disease in a murine model. J. Immunol. 184, 764-774 (2010).
57. Zorn, E. et al. IL-2 regulates FOXP3 expression in human CD4+CD25+ regulatory T cells through a STAT-dependent mechanism and induces the expansion of these cells in vivo. Blood 108, 1571-1579 (2006).
58. Zhao, D. et al. In vivo-activated CD103+CD4+ regulatory T cells ameliorate ongoing chronic graft-versus-host disease. Blood 112, 2129-2138 (2008).
59. Chung, Y. et al. Follicular regulatory T cells expressing Foxp3 and Bcl-6 suppress germinal center reactions. Nat. Med. 17, 983-988 (2011).
60. Wu, H., Xie, M. M., Liu, H. & Dent, A. L. Stat3 is important for follicular regulatory T cell differentiation. PLoS ONE 11, e0155040 (2016).
61. Priceman, S. J. et al. S1PR1 is crucial for accumulation of regulatory T cells in tumors via STAT3. Cell Rep. 6, 992-999 (2014).
62. Nurieva, R. I. et al. Generation of T follicular helper cells is mediated by interleukin-21 but independent of T helper 1, 2, or 17 cell lineages. Immunity 29, 138-149 (2008).
63. Roufaiel, M. et al. CCL19-CCR7-dependent reverse transendothelial migration of myeloid cells clears Chlamydia muridarum from the arterial intima. Nat. Immunol. 17, 1263-1272 (2016).
64. Shaul, M. E., Bennett, G., Strissel, K. J., Greenberg, A. S. & Obin, M. S. Dynamic, M2-like remodeling phenotypes of CD11c+ adipose tissue macrophages during high-fat diet-induced obesity in mice. Diabetes 59, 1171-1181 (2010).
65. Weinstein, J. S. et al. TFH cells progressively differentiate to regulate the germinal center response. Nat. Immunol. 17, 1197-1205 (2016).
66. Wang, H. et al. The transcription factor Foxp1 is a critical negative regulator of the differentiation of follicular helper T cells. Nat. Immunol. 15, 667-675 (2014).
67. Lu, P. et al. Blimp-1 represses CD8 T cell expression of PD-1 using a feedforward transcriptional circuit during acute viral infection. J. Exp. Med. 211, 515-527 (2014).
68. Deng, R. et al. B7H1/CD80 interaction augments PD-1-dependent T cell apoptosis and ameliorates graft-versus-host disease. J. Immunol. 194, 560-574 (2015).
69. Maritano, D. et al. The STAT3 isoforms alpha and beta have unique and specific functions. Nat. Immunol. 5, 401-409 (2004).
70. Anders, S., Pyl, P. T. & Huber, W. HTSeq—a Python framework to work with high-throughput sequencing data. Bioinformatics 31, 166-169 (2015).

1. A method for preventing or treating graft-versus-host disease (GVHD) while preserving graft versus leukemia/lymphoma (GVL) effects in a subject receiving hematopoietic cell transplantation (HCT), comprising administering one or more doses of a therapeutically effective amount of a therapeutic agent to the subject to knock down Stat3 in T cells and/or B cells in vivo.

2. The method of claim 1, wherein the therapeutic agent is administered to the subject simultaneously with HCT, immediately before HCT, or immediately after HCT.

3. The method of claim 1, wherein the GVHD is chronic GVHD.

4. The method of claim 1, wherein the therapeutic agent is a small molecule Stat3 inhibitor delivered by an antibody selected from an anti-CD4 antibody, an anti-CD8 antibody, an anti-CD3 antibody, and an anti-CD19 antibody to specifically knock down Stat3 in the lymphocytes of the target tissues.

5. The method of claim 4, wherein the antibody is a monoclonal antibody, or a humanized antibody.

6. (canceled)

7. The method of claim 4, wherein the small molecule Stat3 inhibitor is a Stat3 siRNA.

8. The method of claim 1, wherein the therapeutic agent specifically targets Stat3 in CD4⁺ T cells.

9. The method of claim 1, further comprising administering thymic progenitors to the subject to reestablish thymus activity in the subject, or one or more doses of Stat3-deficient T cells and/or B cells to the subject.

10. (canceled)

11. The method of claim 9, wherein the Stat3-deficient T cells are Stat3-deficient CD4⁺ T cells.

12. The method of claim 9, wherein the Stat3-deficient T cells and/or B cells are produced in vitro by contacting a population of donor T cells and/or B cells with an effective amount of a therapeutic agent to knock down Stat3 in the donor T cells and/or B cells.

13. The method of claim 12, wherein the therapeutic agent is a small molecule Stat3 inhibitor delivered by an antibody selected from an anti-CD4 antibody, an anti-CD8 antibody, an anti-CD3 antibody, and an anti-CD19 antibody to specifically knock down Stat3 in the lymphocytes of the target tissues.

14. The method of claim 13, wherein the small molecule Stat3 inhibitor is a Stat3 siRNA.

15. A method for preventing or treating graft-versus-host disease (GVHD) while preserving graft versus leukemia/lymphoma (GVL) effects in a subject receiving hematopoietic cell transplantation (HCT), comprising administering one or more doses of Stat3-deficient T cells and/or B cells to the subject.

16. The method of claim 15, wherein Stat3-deficient T cells are Stat3-deficient CD4⁺ T cells.

17-19. (canceled)

20. The method of claim 15, further comprising administering one or more doses of a therapeutically effective amount of a therapeutic agent to the subject to knock down Stat3 in T cells and/or B cells in vivo.

21. The method of claim 16, wherein the therapeutic agent is a small molecule Stat3 inhibitor delivered by an antibody selected from an anti-CD4 antibody, an anti-CD8 antibody, an anti-CD3 antibody, and an anti-CD19 antibody to specifically knock down Stat3 in the lymphocytes of the target tissue.

22. The method of claim 15, further comprising administering thymic progenitors to the subject to reestablish thymus activity in the subject.

23. A method for preventing or treating an autoimmune disease in a subject, comprising:
(i) administering one or more doses of a therapeutically effective amount of a therapeutic agent to the subject to knock down Stat3 in T cells and/or B cells in vivo;
(ii) administering one or more doses of Stat3-deficient donor T cells and/or B cells to the subject; or
(iii) both (i) and (ii).

24. The method of claim 23, wherein the autoimmune disease is Sjogren's syndrome, systemic lupus erythematosus, rheumatoid arthritis, type 1 diabetes, multiple sclerosis, or aplastic anemia.

25. The method of claim 23, wherein the therapeutic agent is a small molecule Stat3 inhibitor delivered by an antibody selected from an anti-CD4 antibody, an anti-CD8 antibody, an anti-CD3 antibody, and an anti-CD19 antibody to specifically knock down Stat3 in the lymphocytes of the target tissue.

26-29. (canceled)


SEQUENCE LISTING (23 sequences)

SEQ ID NO: 1 - DNA, 21 bp, Artificial Sequence
  CCR7 forward primer
tggtggctctccttgtcattt

SEQ ID NO: 2 - DNA, 23 bp, Artificial Sequence
  CCR7 reverse primer
accgactcgtacagggtgtagtc

SEQ ID NO: 3 - DNA, 22 bp, Artificial Sequence
  CXCR4 forward primer
catggaaccgatcagtgtgagt

SEQ ID NO: 4 - DNA, 20 bp, Artificial Sequence
  CXCR4 reverse primer
gcagggttccttgttggagt

SEQ ID NO: 5 - DNA, 22 bp, Artificial Sequence
  CXCR5 forward primer
actccttaccacagtgcacctt

SEQ ID NO: 6 - DNA, 19 bp, Artificial Sequence
  CXCR5 reverse primer
ggaaacgggaggtgaacca

SEQ ID NO: 7 - DNA, 20 bp, Artificial Sequence
  BCL6 forward primer
cacacccgtccatcattgaa

SEQ ID NO: 8 - DNA, 20 bp, Artificial Sequence
  BCL6 reverse primer
tgtcctcacggtgccttttt

SEQ ID NO: 9 - DNA, 21 bp, Artificial Sequence
  ICOS forward primer
ctcaccaagaccaagggaagc

SEQ ID NO: 10 - DNA, 21 bp, Artificial Sequence
  ICOS reverse primer
ccacaacgaaagctgcacacc

SEQ ID NO: 11 - DNA, 20 bp, Artificial Sequence
  PDCD-1 forward primer
cgtccctcagtcaagaggag

SEQ ID NO: 12 - DNA, 20 bp, Artificial Sequence
  PDCD-1 reverse primer
gtccctagaagtgcccaaca

SEQ ID NO: 13 - DNA, 20 bp, Artificial Sequence
  CD80 forward primer
ggcaaggcagcaatacctta

SEQ ID NO: 14 - DNA, 20 bp, Artificial Sequence
  CD80 reverse primer
ctctttgtgctgctgattcg

SEQ ID NO: 15 - DNA, 26 bp, Artificial Sequence
  PD-L1 forward primer
aggatatttgctggcattatattcac

SEQ ID NO: 16 - DNA, 25 bp, Artificial Sequence
  PD-L1 reverse primer
acaaactgaatcacttgctcatctt

SEQ ID NO: 17 - DNA, 19 bp, Artificial Sequence
  GAPDH forward primer
tcaccaccatggagaaggc

SEQ ID NO: 18 - DNA, 20 bp, Artificial Sequence
  GAPDH reverse primer
gctaagcagttggtggtgca

SEQ ID NO: 19 - DNA, 20 bp, Artificial Sequence
  shSTAT3-1 target sequence
gtgtcagatcacatgggcta

SEQ ID NO: 20 - RNA, 20 nt, Artificial Sequence
  shSTAT3-1 antisense target sequence
uagcccaugugaucugacac

SEQ ID NO: 21 - DNA, 20 bp, Artificial Sequence
  shSTAT3-2 target sequence
gcagctgaacaacatgtcat

SEQ ID NO: 22 - RNA, 20 nt, Artificial Sequence
  shSTAT3-2 antisense sequence
augacauguuguucagcugc

SEQ ID NO: 23 - RNA, 22 nt, Artificial Sequence
  shRNA control sequence
agcguucuacacucgacguacu